(19) United States
(12) Patent Application Publication
(10) Pub. No.: US 2010/0184722 A1
(43) Pub. Date: Jul. 22, 2010

(54) INCLUSION COMPLEXES OF ALPHA-CYCLODEXTRIN AND SILDENAFIL SALT

(75) Inventors: Henk Swart, George (ZA); Steyn Knoetze, Sedgefield (ZA)

Correspondence Address: MORRISON & FOERSTER LLP, 755 PAGE MILL RD, PALO ALTO, CA, 94304-1018, US

(73) Assignee: Shimoda Biotech (Pty) Ltd, Port Elizabeth (ZA)

(21) Appl. No.: 12/642,742

(22) Filed: Dec. 18, 2009

Related U.S. Application Data
(60) Provisional application No. 61/139,471, filed on Dec. 19, 2008.

(51) Int. Cl. A61K 31/724; C08B 37/16; A61K 31/519; A61P 9/12; A61P 15/10; C12N 9/99

(52) U.S. Cl. 514/58; 536/103; 514/252.16; 435/184

(57) ABSTRACT

Provided are inclusion complexes comprising a sildenafil salt (e.g., sildenafil citrate) and alpha-cyclodextrin. The complexes may be useful treating various conditions, such as male erectile dysfunction and pulmonary hypertension. In some instances the inclusion complexes increase the solubility of sildenafil. Also provided are methods of producing the inclusion complexes, as well as methods of treatment, kits and unit dosages.

### CROSS-REFERENCE TO RELATED APPLICATIONS

This application claims priority to U.S. Provisional Patent Application No. 61/139,471 filed Dec. 19, 2008, the disclosure of which is hereby incorporated herein by reference in its entirety.

### FIELD OF THE INVENTION

The present invention relates to formulations and inclusion complexes between alpha-cyclodextrin and sildenafil salts, to methods of preparing such inclusion complexes, and methods of increasing the water solubility of sildenafil and sildenafil salts. Moreover, the present invention relates to the use of alpha-cyclodextrin-sildenafil salt inclusion complexes and pharmaceutical formulations for use in the treatment of, for example, male erectile dysfunction and pulmonary hypertension.

### BACKGROUND OF THE INVENTION

Sildenafil base, also known as 5-[2-ethoxy-5-(4-methylpiperazin-1-yl)sulfonylphenyl]-1-methyl-3-propyl-4H-pyrazolo[5,4-e]pyrimidin-7-one or 1-[4-ethoxy-3-(6,7-dihydro-1-methyl-7-oxo-3-propyl-1H-pyrazolo[4,3-d]pyrimidin-5-yl)phenylsulfonyl]-4-methyl-piperazine, and its salts, particularly sildenafil citrate, also known as 1-[[3-(6,7-dihydro-1-methyl-7-oxo-3-propyl-1H15-pyrazolo[4,3-d]pyrimidin-5-yl)-4-ethoxyphenyl]sulfonyl]-4-methylpiperazine citrate, are classified as selective inhibitors of cyclic guanosine monophosphate (cGMP)-specific phosphodiesterase 12 type-5 (PDE5). Sildenafil citrate is commonly used in the treatment of erectile dysfunction in the approved formulation Viagra® (see U.S. Pat. No. 5,250,534 and U.S. Pat. No. 6,469,012). Sildenafil and salts thereof may also be effective in the treatment of, for example, female sexual arousal disorder, pulmonary hypertension, Rynaude's Phenomenon and altitude sickness.

In the treatment of certain conditions, such as sexual dysfunction (e.g., male erectile dysfunction or female sexual arousal disorder) a rapid absorption and achievement of drug concentration along with a greater prospect for rapid onset of therapeutic effect, frequently is sought by individuals desiring more immediate and/or less prolonged effects. An important factor affecting the absorption of a pharmaceutical agent is its solubility and rate of dissolution. Accordingly, there is a need to increase the solubility and/or rate of dissolution for poorly soluble drugs such as Sildenafil.

Cyclodextrins have been used to influence the solubility properties of various compounds by forming inclusion complexes (see Szejtli, J. Cyclodextrin Technology (1988) Kluwer Academic Publishers, Dordrecht). Briefly, cyclodextrins are commercially available cyclic oligosaccharides composed of 6, 7 or 8 glucopyranose units (alpha-, beta- and gamma-cyclodextrin, respectively) characterized by a cone-like molecular shape. The cavity of the cone is hydrophobic whilst the exterior is hydrophilic. The hydrophobic nature of the cavity, in certain cases, endows the cyclodextrin with the ability to form inclusion complexes with hydrophobic guest molecules of suitable size to fit into the cavity of the host. The inclusion complex may be stabilized by a number of forces, such as van der Waals attractive forces, electrostatics and hydrogen bonding. Polar and ionic groups are generally less likely to be included within the hydrophobic cavity than less-polar and non-ionic groups. In addition to influencing solubility, cyclodextrin inclusion complexes, in some cases, may provide favorable flow, binding, and compaction properties to aid in drug formulation (e.g., in facilitating tablet compression).

Guest-host interactions between sildenafil base and a group of cyclodextrins, including alpha-cyclodextrin (ACD); beta-cyclodextrin (BCD); gamma-cyclodextrin (GCD) and hydroxypropyl-beta-cyclodextrin (HPBCD) were evaluated by Omari, et al. (2006). Their investigations employed several techniques, including phase solubility diagrams (PSD), differential scanning calorimetry (DSC), X-ray powder diffractometry (XRPD), proton nuclear magnetic resonance (1H NMR) and molecular mechanical modeling (MM+). Estimates of the complex formation constant (K₁₁) show that the tendency of sildenafil base to complex with the selected cyclodextrins, follows the order of BCD>HPBCD>GCD, ACD. The low formation complex observed for ACD was attributed to high water solubility of the cyclodextrin and a resultant lower complexation driving force and a small cavity size, reducing the probability of including the bulky groups of sildenafil. Aqueous solubility studies showed that the sildenafil solubility was enhanced in the following order BCD>HPBCD>GCD>ACD. These results teach that ACD resulted in the lowest solubility enhancement of sildenafil base, with BCD being significantly superior. The authors conclude that the data shows that BCD indicates a better geometric fit than the ACD or GCD cavities. Moreover, the authors suggest that the higher inherent solubilities of ACD, HPBCD and GCD in water tend to lower their affinities for to hydrophobic substrates.

Sildenafil formulations have been described in, for example, CA2346350, U.S. Pat. No. 6,087,362, WO0135926, EP1514877. However, a need in the art continues to exist for improved solubility and rate of dissolution properties for poorly soluble drugs such as Sildenafil.

The disclosures of all publications, patents, patent applications and other references referred to herein are hereby incorporated herein by reference in their entireties.

### BRIEF SUMMARY OF THE INVENTION

One aspect described herein provides an inclusion complex comprising a sildenafil salt and an optionally substituted alpha-cyclodextrin. In one embodiment, the sildenafil salt is an organic salt (e.g., sildenafil mesylate or sildenafil-citrate). In one embodiment, the sildenafil salt is sildenafil-citrate.

In some of these embodiments, the molar ratio of the sildenafil salt to alpha-cyclodextrin is from about 1:1 to 1:20, or 1:1 to 1:5, or 1:1 to 1:2.5, inclusive.

In some of these embodiments of the inclusion complex, the solubility of the sildenafil salt upon dissolution of the complex in deionized water at 20° C. is increased by at least about 1.5-fold, or about 2-fold, when compared to the solubility of sildenafil salt in uncomplexed form.

In some of these embodiments of the inclusion complex, the solubility of the sildenafil salt upon dissolution of the complex in deionized water at 20° C. is at least about 8 mM, or about 10 mM, or about 12 mM.

In another aspect is provided a formulation comprising a sildenafil salt, alpha-cyclodextrin, and a carrier. In some embodiments, the formulation comprises an effective amount a sildenafil salt, alpha-cyclodextrin, and a carrier. In some embodiments, the molar ratio of alpha-cyclodextrin to sildenafil salt is greater than about 1:1, or greater than about 5:1, or greater than about 10:1, inclusive.

In another aspect is provided a formulation comprising an inclusion complex described herein and a carrier. In some embodiments, the formulation comprises an effective amount of the inclusion complex and a carrier.

In some of embodiments of the formulations described herein, the carrier is a pharmaceutically acceptable carrier. In some of these embodiments, the formulation is a solid. In some embodiments, the formulation is a liquid. In some embodiments, the pH of the liquid is less than about 8.0 at 20° C., or between about 4.0 and 6.0 at 20° C.

In some embodiments of the formulations described herein, the sildenafil salt is present in an amount between about 0.1 mg and about 200 mg, or between about 20 mg and about 100 mg, inclusive, or about 25 mg, or 50 mg, or 75 mg, 100 mg, or 150 mg. In some embodiments of the formulations described herein, the sildenafil salt is present in an amount between about 0.1 mg and about 200 mg sildenafil, or between about 20 mg and about 100 mg sildenafil, inclusive, or about 25 mg, or 50 mg, or 75 mg, 100 mg, or 150 mg sildenafil.

In another aspect, the inclusion complex described herein is in a substantially pure form.

In another aspect, are provided methods of treating erectile dysfunction in an individual, comprising administering to the individual an effective amount of an inclusion complex of a sildenafil salt and alpha-cyclodextrin, or a formulation thereof.

In another aspect, are provided methods of treating erectile dysfunction in an individual, comprising administering to the individual an effective amount of a formulation comprising a sildenafil salt, alpha-cyclodextrin, and a carrier (e.g., a pharmaceutically acceptable carrier).

In another aspect, are provided methods of treating pulmonary hypertension in an individual, comprising administering to the individual an effective amount of an inclusion complex of a sildenafil salt and alpha-cyclodextrin, or a formulation thereof.

In another aspect, are provided methods of treating pulmonary hypertension in an individual, comprising administering to the individual an effective amount of a formulation comprising a sildenafil salt, alpha-cyclodextrin, and a carrier (e.g., a pharmaceutically acceptable carrier). In some embodiments, the pulmonary hypertension is neonatal pulmonary hypertension and the individual is a neonate.

In some embodiments of the methods, the complex and/or formulation is administered parenterally. In some embodiments, the complex and/or formulation is administered orally. In some embodiments, the dosage of sildenafil salt is between about 0.1 mg and about 200 mg, or between about 20 mg and about 100 mg, inclusive, or about 25 mg, or about 50 mg, or about 100 mg. In some embodiments, the dosage of sildenafil salt is between about 0.1 mg and about 200 mg sildenafil, or between about 20 mg and about 100 mg sildenafil, inclusive, or about 25 mg, or about 50 mg, or about 100 mg sildenafil. In some embodiments, the individual is a human (e.g., an adult or a neonate).

In another aspect, are provided methods of inhibiting cyclic guanosine monophosphate (cGMP)-specific phosphodiesterase 12 type-5 (PDE5), comprising contacting the PDE5 enzyme with an effective amount of the sildenafil salt of an inclusion complex described herein.

In another aspect, is provided an inclusion complex of a sildenafil salt and alpha-cyclodextrin for use in a method of treating erectile dysfunction or pulmonary hypertension in an individual.

In another aspect, is provided the use of the inclusion complex of a sildenafil salt and alpha-cyclodextrin for the manufacture of a medicament for use in a method of treating erectile dysfunction or pulmonary hypertension in an individual.

In another aspect, is provided a kit for the treatment of erectile dysfunction or pulmonary hypertension, comprising an inclusion complex of a sildenafil salt and alpha-cyclodextrin or formulation thereof; and instructions for use.

In another aspect, are provided methods of producing an inclusion complex of a sildenafil salt and alpha-cyclodextrin, comprising admixing the sildenafil salt with alpha-cyclodextrin. In some embodiments, the method further comprises adding a solvent, mixed solvent, or buffer to the sildenafil salt, alpha-cyclodextrin, and/or mixture thereof.

In another aspect, are provided methods of producing an inclusion complex of a sildenafil salt and alpha-cyclodextrin, comprising the steps of: (a) admixing the sildenafil salt and alpha-cyclodextrin; and (b) adding a suitable amount of solvent, mixed solvent, or buffer to the mixture of step (a) and mixing until a suspension or solution is formed.

In some of these embodiments, the solvent, mixed solvent, or buffer is a buffer. In some embodiments, the buffer is a phosphate-citrate buffer. In some embodiments, the buffer has a pH between about 4.0 and about 6.0, or a pH of about 5.0.

In some embodiments of the methods of producing an inclusion complex, the solvent, mixed solvent, or buffer is heated to greater than about 40° C., or greater than about 50° C., or greater than about 60° C.

In some embodiments of the methods of producing an inclusion complex, step (a) further comprises admixing a suitable polymer. In some embodiments, the suitable polymer is selected from polyvinylpyrrolidone, hydroxypropyl methylcellulose, carboxymethylcellulose, and Plasdone® Povidone.

In some embodiments of the methods of producing an inclusion complex, the mixing is continued for at least about 0.2 hr, or about 0.5 hr following formation of the suspension or solution.

In some embodiments, the methods of producing an inclusion complex further comprise the step of: (c) drying the product of step (b). In some embodiments, the drying comprises evaporation. In some embodiments, the evaporation occurs for greater than about 1 hour. In some embodiments, the evaporation is conducted under vacuum. In some embodiments, the evaporation is conducted under atmospheric pressure. In some embodiments, the drying comprises spray-drying. In some embodiments, the drying comprises freeze-drying. In some embodiments, the drying comprises spray-granulation.

In another aspect, are provided methods for improving the solubility of a sildenafil salt in water comprising complexing the sildenafil salt with alpha-cyclodextrin. In some embodiments, the sildenafil salt is sildenafil citrate. In some embodiments, the sildenafil salt in deionized water at 20° C. is increased by at least about 1.5-fold or about 2-fold compared to the solubility of sildenafil salt in uncomplexed form.

### BRIEF DESCRIPTION OF THE FIGURES

FIG. 1A shows solubility data for sildenafil citrate in the presence of various concentrations of cyclodextrins (alpha-cyclodextrin (0-100 mg/ml); gamma-cyclodextrin 0-200 mg/ml) and hydroxypropyl beta-cyclodextrin (0-200 mg/ml).

FIG. 1B shows solubility data for sildenafil citrate in the presence of beta-cyclodextrin (0-15 mg/ml).

FIG. 2A shows solubility data for sildenafil base in the presence of various concentrations of cyclodextrins (alpha-cyclodextrin (0-100 mg/ml); gamma-cyclodextrin 0-200 mg/ml) and hydroxypropyl beta-cyclodextrin (0-200 mg/ml).

FIG. 2B shows solubility data for sildenafil base in the presence of beta-cyclodextrin (0-15 mg/ml).

FIG. 3 shows solubility data for sildenafil citrate in the absence and presence of two cyclodextrins (alpha-cyclodextrin (100 mg/ml) and hydroxypropyl beta-cyclodextrin (200 mg/ml) in phosphate-citrate buffer (0.1M) over a pH range of 3.0-8.0.

FIG. 4 shows comparative dissolution rates of tablets containing sildenafil citrate and alpha-cyclodextrin as an inclusion complex or a physical mixture.

FIG. 5 shows sildenafil concentration vs. time for orally administered solutions of sildenafil citrate and alpha-cyclodextrin compared to a sildenafil citrate suspension in a rat model.

### DETAILED DESCRIPTION OF THE INVENTION

Provided herein are inclusion complexes comprising a sildenafil salt and alpha-cyclodextrin. Surprisingly, the inventors have found that these complexes result in remarkable solubility enhancement of sildenafil, and a greater solubility enhancement when compared to sildenafil base and sildenafil salts complexed with other cyclodextrins. The inventors have also found a striking increased rate of dissolution and absorption profile for the sildenafil salt of the alpha-cyclodextrin complex compared to the uncomplexed sildenafil salt. Such complexes may provide a more rapid onset of therapeutic effect and/or less prolonged effects for the treatment of certain conditions, such as conditions related to cGMP-specific phosphodiesterase type 5 (PDE5). These inclusion complexes may be particularly useful for the treatment of conditions such as sexual dysfunction (e.g., male erectile dysfunction) and pulmonary hypertension.

In one aspect, are provided sildenafil salt formulations and sildenafil salt inclusion complexes as described herein. In another aspect, are provided methods of preparing the inclusion complexes. In another aspect, are provided methods of treating a condition (e.g., erectile dysfunction or pulmonary hypertension) using the formulations or inclusion complexes described herein. Also provided are kits and unit dosage forms of the sildenafil salt inclusion complexes.

### ABBREVIATIONS AND DEFINITIONS

With reference to cyclodextrin, as used herein, the term “inclusion complex” is intended a complex wherein a moiety of a compound (e.g., sildenafil) is inserted, at least partially, into the cavity of a cyclodextrin (e.g., alpha-cyclodextrin). The inserted compound of the inclusion complex is considered “complexed” while the compound alone is considered “uncomplexed.”

As used herein, the term “solubility” intends the solubility with reference to the total amount of compound (e.g., including the amount of compound in both complexed and uncomplexed form).

The term “sildenafil base,” as used herein refers to sildenafil in a non-salt form.

As used herein, “treatment”, “treating”, or “treat” is an approach for obtaining beneficial or desired results, including clinical results. For purposes herein, beneficial or desired results include, but are not limited to, one or more of the following: decreasing one more symptoms resulting from the condition (e.g., erectile dysfunction or pulmonary hypertension), diminishing the extent of the disease, stabilizing the condition, delaying or slowing the progression of the condition, reversing the progression or severity of the condition, ameliorating the condition, decreasing the dose of one or more other medications required to treat the condition, and/or increasing the quality of life of an individual who has been or is suspected of having the condition. The methods described herein contemplate any one or more of these aspects of treatment.

With respect to a condition, “delaying” means to defer, hinder, slow, retard, stabilize, and/or postpone development of, and/or one or more symptoms of the condition (e.g., erectile dysfunction or pulmonary hypertension). This delay can be of varying lengths of time, depending on the history of the disease and/or individual being treated. As is evident to one skilled in the art, a sufficient or significant delay can, in effect, encompass prevention, in that the individual does not develop the condition (e.g., erectile dysfunction or pulmonary hypertension). A method that “delays” development of the condition is a method that reduces the probability of development in a given time frame and/or reduces the extent of the condition in a given time frame, when compared to not using the method. Such comparisons are typically based on clinical studies, using a statistically significant number of subjects.

As used herein, “pharmaceutically acceptable” with respect to a material, refers to a material that is not biologically or otherwise unsuitable, e.g., the material may be incorporated (e.g., at the time of manufacturing or administration) into a pharmaceutical composition administered to an individual without causing any significant undesirable biological effects or interacting in a deleterious manner with any of the other components of the composition in which it is contained. As used herein, the term “pharmaceutically acceptable carrier,” refers to, for example, solvents, stabilizers, pH-modifiers, tonicity modifiers, adjuvants, binders, diluents, complexing agents, fillers, granulating agents, disintegrants, lubricants, glidants, etc., known to the skilled artisan that are suitable for administration to an individual (e.g., a human). Combinations of two or more carriers are also contemplated. The pharmaceutically acceptable carrier(s) and any additional components, as described herein, should be compatible for use in the intended route of administration (e.g., oral, parenteral) for a particular dosage form. Such suitability will be easily recognized by the skilled artisan, particularly in view of the teaching provided herein. Pharmaceutically acceptable carriers or excipients have preferably met the required standards of toxicological and manufacturing testing and/or are included on the Inactive Ingredient Guide prepared by the U.S. Food and Drug administration.

With respect to treatment, an “effective amount,” as used herein refers to an amount that results in a desired pharmacological and/or physiological effect for a specified condition (e.g., sexual dysfunction or pulmonary hypertension) or one or more of its symptoms and/or to completely or partially prevent the occurrence or recurrence of the condition or symptom thereof and/or may be therapeutic in terms of a partial or complete cure for the condition and/or adverse effect attributable to the condition (e.g., sexual dysfunction or pulmonary hypertension). In reference to conditions described herein (e.g., sexual dysfunction or pulmonary hypertension), an effective amount may comprise an amount sufficient to, among other things, provide and sustain an erection in a male (e.g., via an increase in intracavernosal pressure leading to an erection sufficient for intercourse) or enhance the ability of a female to achieve or sustain an aroused state in the case of sexual dysfunction; or reduce elevated pulmonary vascular resistance in the case of pulmonary hypertension. In certain embodiments, the pharmaceutically effective amount is sufficient to prevent the condition, as in being administered to an individual prophylactically. Effective amount includes the eradication or amelioration of the underlying condition being treated and/or eradication or amelioration of one or more of the symptoms associated with the underlying condition such that the individual reports an improvement in feeling or condition (e.g., increased sensitivity to sexual stimuli), notwithstanding that the individual may still be afflicted with the underlying condition. Effective amount also includes halting or slowing the progression of the condition (e.g., erectile dysfunction or pulmonary hypertension), regardless of whether improvement of the condition is realized.

The “effective amount” may vary depending on the composition being administered, the condition being treated/prevented, the severity of the condition being treated/prevented, the age, body size, weight, and relative health of the individual, the route and form of administration, the judgment of the attending medical or veterinary practitioner (if applicable), and other factors appreciated by the skilled artisan in view of the teaching provided herein. An effective amount may be assessed, for example, by using data from one or more clinical (e.g., penile plethysmography), physiological, biochemical, histological, electrophysiological, and/or behavioral evaluations.

As is understood in the art, an “effective amount” may be in one or more doses, i.e., a single dose or multiple doses may be required to achieve the desired treatment endpoint. An effective amount may be considered in the context of administering one or more additional pharmaceutical agents, and a sildenafil salt/alpha-cyclodextrin inclusion complex may be considered to be given in an effective amount if, in conjunction with one or more additional pharmaceutical agents, one or more desirable or beneficial result(s) may be or are achieved.

When used with respect to methods of treatment/prevention and the use of the sildenafil salt/alpha-cyclodextrin inclusion complex and formulations thereof described herein, an individual “in need thereof” may be an individual who has been diagnosed with, previously treated for, and/or suspected of having the condition to be treated (e.g., erectile dysfunction or pulmonary hypertension). With respect to prevention, the individual in need thereof may also be an individual who is at risk for a condition (e.g., age, a family history of the condition, life-style factors indicative of risk for the condition, etc.).

In some embodiments, the individual is a mammal, including, but not limited to, bovine, horse, feline, rabbit, canine, rodent, or primate. In some embodiments, the mammal is a primate. In some embodiments, the primate is a human. In some embodiments, the individual is human, including adults, children, infants, and preemies. In some embodiments, the individual is a non-mammal. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like. In some embodiments, the individual is a non-mammal, including, but not limited to, birds, and the like. The term “individual” does not denote a particular age or sex.

With respect to sildenafil, “combination therapy” means a first therapy that includes sildenafil (e.g., as a sildenafil salt in a formulation comprising alpha-cyclodextrin and/or as an inclusion complex of a sildenafil salt with alpha-cyclodextrin), in conjunction with a second therapy (e.g., surgery and/or an additional pharmaceutical agent) useful for treating, stabilizing, preventing, and/or delaying a disease or condition. Administration in “conjunction with” another compound includes administration in the same or different composition(s), either sequentially, simultaneously, or continuously, through the same or different routes. In some embodiments, the combination therapy optionally includes one or more pharmaceutically acceptable carriers or excipients, non-pharmaceutically active compounds, and/or inert substances.

With respect to sildenafil, the term “additional pharmaceutical agent,” refers to an active agent other than sildenafil, for example, a drug, which is administered to elicit a therapeutic effect. The pharmaceutical agent(s) may be directed to a therapeutic effect related to one or more conditions that sildenafil is intended to treat or prevent (e.g., erectile dysfunction or pulmonary hypertension) or the pharmaceutical agent may be intended to treat or prevent a symptom of the underlying condition (e.g., shortness of breath, fatigue, cough, angina pectoris, fainting or syncope, peripheral edema, hemoptysis, etc. in the case of pulmonary hypertension) or to further reduce the appearance or severity of side effects of sildenafil.

Reference to “about” a value or parameter herein includes (and describes) variations that are directed to that value or parameter per se. For example, a description referring to “about X” includes the description of “X”.

As used herein and in the appended claims, the singular forms “a,” “or,” and the include plural referents unless the context clearly dictates otherwise. It is understood that aspect and variations described herein include “consisting” and/or “consisting essentially of” aspects and variations.

Unless defined otherwise or clearly indicated by context, all technical and scientific terms and abbreviations used herein have the same meaning as commonly understood by one of ordinary skill in the art to which this invention belongs.

### Inclusion Complexes

Described herein are inclusion complexes containing a sildenafil salt and alpha-cyclodextrin which may be useful in the treatment of conditions (e.g., sexual dysfunction and/or pulmonary hypertension).

In one aspect the inclusion complex comprises a sildenafil salt and an optionally substituted alpha-cyclodextrin. The sildenafil salt may be any pharmaceutically acceptable salt. “Pharmaceutically acceptable salts” are those salts which retain the biological activity of the sildenafil base and which can be administered as drugs or pharmaceuticals to and individual (e.g., a human). In some embodiments, the sildenafil salt comprises an organic salt (e.g., citrate, mesylate, acetate, maleate, fumarate, succinate, L-ascorbate, 2-hydroxypropanoate, tartrate). In some embodiments, the sildenafil salt is sildenafil citrate. In other embodiments, the sildenafil salt comprises an inorganic salt (e.g., HCl, HBr, and nitrate).

The inclusion complex may comprise a sildenafil salt that is partially or completely included into the cavity of the alpha-cyclodextrin molecule. Accordingly, one or more alpha-cyclodextrin molecules may be associated with each sildenafil salt molecule. The complex may exist in a variety of molar ratios which may be dependent on a variety of physical factors during the formation of the complex, as well as the selection of sildenafil salt for inclusion. The molar ratio of the inclusion complex also may be transitional and vary during formation.

In some embodiments, the sildenafil salt (e.g., sildenafil citrate) is fully included into the cavity of an alpha-cyclodextrin molecule. In some embodiments, the sildenafil salt (e.g., sildenafil citrate) is partially included into the cavity of an alpha-cyclodextrin molecule. In some embodiments of the inclusion complex, the molar ratio of the sildenafil salt (e.g., sildenafil citrate) to alpha-cyclodextrin is from 1:1 to 1:40, 1:1 to 1:30, 1:1 to 1:25, 1:1 to 1:20, 1:1 to 1:15, 1:1 to 1:10, 1:1 to 1:5, 1:1 to 1:4, 1:1 to 1:3, 1:1 to 1:2.5, 1:1 to 1:2, inclusive, or 1:1, 1:2, or 1:3.

The inclusion complexes described herein may increase the solubility of sildenafil. In some embodiments, the solubility of the sildenafil salt (e.g., sildenafil citrate) upon dissolution of the inclusion complex (e.g., a complex comprising sildenafil citrate and alpha-cyclodextrin) in deionized water at 20° C. is increased by at least 1.25-fold compared to the solubility of sildenafil salt in uncomplexed form. In some embodiments, the solubility is increased by at least any of about 1.5-, 1.75-, 2-, 2.25-, 2.5-, 2.75-, 3-, 3.25-, 3.5-, 3.75-, 4-, 5-, 7.5-, or 10-fold. In another aspect, are provided methods for improving the solubility of a sildenafil salt (e.g., sildenafil citrate) in water comprising complexing the sildenafil salt with alpha-cyclodextrin. In some embodiments, the sildenafil salt is sildenafil citrate. In some embodiments, the sildenafil salt in deionized water at 20° C. is increased by at least any of about 1.25-, 1.5-, 1.75-, 2-, 2.25-, 2.5-, 2.75-, 3-, 3.25-, 3.5-, 3.75-, 4-, 5-, 7.5-, or 10-fold compared to the solubility of sildenafil salt in uncomplexed form.

In some embodiments, the solubility of the sildenafil salt (e.g., sildenafil citrate) upon dissolution of the inclusion complex (e.g., a complex comprising sildenafil citrate and alpha-cyclodextrin) in deionized water at 20° C. is at least about 10-fold greater than the solubility of sildenafil base in uncomplexed form. In some embodiments, the solubility is increased by at least any of about 10-, 25-, 50-, 75-, or 100-fold. In another aspect, are provided methods for improving the solubility of a sildenafil base in water comprising converting the sildenafil base to a sildenafil salt (e.g., sildenafil citrate) and complexing the sildenafil salt with alpha-cyclodextrin. In some embodiments, the solubility of sildenafil (in the form of a sildenafil salt) in deionized water at 20° C. is increased by at least any of about 10-, 25-, 50-, 75-, or 100-fold compared to the solubility of sildenafil base in uncomplexed form.

In some embodiments, the solubility of the sildenafil salt (e.g., sildenafil citrate) upon dissolution of the inclusion complex (e.g., a complex comprising sildenafil citrate and alpha-cyclodextrin) in deionized water at 20° C. is at least any of about 4 mM, 6 mM, 8 mM, 10 mM, 12 mM, 15 mM, 20 mM, or 25 mM.

The inclusion complexes described herein may provide improved pharmacokinetic properties for sildenafil. Such changes in pharmacokinetic properties may result in desired therapeutic effects, such as a more rapid onset of therapeutic effect and/or less prolonged effects for the treatment of certain conditions (e.g., erectile dysfunction and pulmonary hypertension).

The inclusion complexes described herein may result in increased oral bioavailability for sildenafil. In one embodiment, the oral bioavailability of sildenafil from an inclusion complex comprising sildenafil salt (e.g., sildenafil citrate) and alpha-cyclodextrin is at least any of about 2%, 5%, 10%, 15%, 20%, 25%, 30%, 35%, 40%, 45%, 50%, 60%, 70%, 80%, or 90% greater than the oral bioavailability of sildenafil salt alone (e.g., sildenafil citrate alone) under the same conditions. In some embodiments are provided methods of increasing oral bioavailability of a sildenafil salt (e.g., sildenafil citrate) by at least any of about 2%, 5%, 10%, 15%, 20%, 25%, 30%, 35%, 40%, 45%, 50%, 60%, 70%, 80%, or 90% comprising administering the sildenafil salt in the form of an inclusion complex comprising sildenafil salt (e.g., sildenafil citrate) and alpha-cyclodextrin under the same conditions. Bioavailability may be determined using standard techniques known in the art (e.g., measuring AUC(oral)/AUC(injected)×100). In some of these embodiments, the conditions comprise orally administering any of about 5, 10, 15, 20 or 25 mg/mL of the sildenafil salt (in the appropriate complexed or uncomplexed form) at room temperature in a buffered solution (e.g., using phosphate-citrate buffer) at about pH 5.

The inclusion complexes described herein may result in an increased Cₘₐₓ (maximum concentration) for sildenafil following administration. In one embodiment, the Cₘₐₓ of sildenafil from an inclusion complex comprising sildenafil salt (e.g., sildenafil citrate) and alpha-cyclodextrin is at least any of about 2%, 5%, 10%, 15%, 20%, 25%, 30%, 35%, 40%, 45%, 50%, 60%, 70%, 80%, or 90% greater than the Cₘₐₓ of sildenafil salt alone (e.g., sildenafil citrate alone) under the same conditions. In some embodiments are provided methods of increasing the Cₘₐₓ of a sildenafil salt (e.g., sildenafil citrate) by at least any of about 2%, 5%, 10%, 15%, 20%, 25%, 30%, 35%, 40%, 45%, 50%, 60%, 70%, 80%, or 90% comprising administering the sildenafil salt in the form of an inclusion complex comprising sildenafil salt (e.g., sildenafil citrate) and alpha-cyclodextrin under the same conditions. In some of these embodiments, the conditions comprise orally administering any of about 0.1, 0.5, 1, 2, 5, 10, 15, 20 or 25 mg/mL of the sildenafil salt (in the appropriate complexed or uncomplexed form) at room temperature in a buffered solution (e.g., using phosphate-citrate buffer) at about pH 5.

The inclusion complexes described herein may result in a decreased Tₘₐₓ (time to reach maximum concentration) for sildenafil following administration. In one embodiment, the Tₘₐₓ of sildenafil from an inclusion complex comprising sildenafil salt (e.g., sildenafil citrate) and alpha-cyclodextrin is at least any of about 2%, 5%, 10%, 15%, 20%, 25%, 30%, 35%, 40%, 45%, 50%, 60%, 70%, 80%, or 90% lower than the Tₘₐₓ of sildenafil salt alone (e.g., sildenafil citrate alone) under the same conditions. In some embodiments are provided methods of decreasing the Tₘₐₓ of a sildenafil salt (e.g., sildenafil citrate) by at least any of about 2%, 5%, 10%, 15%, 20%, 25%, 30%, 35%, 40%, 45%, 50%, 60%, 70%, 80%, or 90% comprising administering the sildenafil salt in the form of an inclusion complex comprising sildenafil salt (e.g., sildenafil citrate) and alpha-cyclodextrin under the same conditions. In some of these embodiments, the conditions comprise orally administering any of about 0.1, 0.5, 1, 2, 5, 10, 15, 20 or 25 mg/mL of the sildenafil salt (in the appropriate complexed or uncomplexed form) at room temperature in a buffered solution (e.g., using phosphate-citrate buffer) at about pH 5.

The inclusion complexes described herein may result in decreasing the therapeutic time of onset for sildenafil. In one embodiment, the therapeutic time of onset of sildenafil from an inclusion complex comprising sildenafil salt (e.g., sildenafil citrate) and alpha-cyclodextrin is decreased by at least any of about 2%, 5%, 10%, 15%, 20%, 25%, 30%, 35%, 40%, 45%, 50%, 60%, 70%, 80%, or 90% than the therapeutic time of onset of sildenafil salt alone (e.g., sildenafil citrate alone) under the same conditions. In some embodiments are provided methods of decreasing the therapeutic time of onset of a sildenafil salt (e.g., sildenafil citrate) by at least any of about 2%, 5%, 10%, 15%, 20%, 25%, 30%, 35%, 40%, 45%, 50%, 60%, 70%, 80%, or 90% comprising administering the sildenafil salt in the form of an inclusion complex comprising sildenafil salt (e.g., sildenafil citrate) and alpha-cyclodextrin under the same conditions. In some of these embodiments, the conditions comprise orally administering any of about 0.1, 0.5, 1, 2, 5, 10, 15, 20 or 25 mg/mL of the sildenafil salt (in the appropriate complexed or uncomplexed form) at room temperature in a buffered solution (e.g., using phosphate-citrate buffer) at about pH 5.

In some embodiments, the inclusion complex comprising a sildenafil salt (e.g., sildenafil citrate) and alpha-cyclodextrin is in substantially pure form. Unless otherwise stated, “substantially pure” intends a preparation of the inclusion complex that contains no more than 15% impurity, wherein the impurity intends a compound other than the sildenafil salt and alpha-cyclodextrin. As used in the context of substantially pure, impurity also intends sildenafil base. In one variation, a preparation of substantially pure inclusion complex is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

The inclusion complexes described herein, formulations thereof, and methods include all solvate and/or hydrate forms. In some embodiments, the inclusion complexes described herein can exist in unsolvated forms as well as solvated forms (i.e., solvates). The inclusion complexes may also include hydrated forms (i.e., hydrates).

### Methods of Preparation

Also provided are methods of preparing the inclusion complexes described herein. In some instances inclusion complexes may be prepared on the basis of liquid state, solid state or semi-solid state reaction between the components. The former is accomplished by dissolving the cyclodextrin and guest in a suitable solvent or mixture of solvents and subsequently isolating the solid state complex by crystallization, evaporation, spray drying or freeze drying. In the solid state method, the two components may be screened to uniform particle size and thoroughly mixed, at which point they may be ground in a high energy mill with optional heating, screened and homogenized. In the semi-solid state, the two components are kneaded in the presence of small amounts of a suitable solvent, and the complex so-formed, is oven dried, screened and homogenized. The liquid state reaction generally provides optimum conditions for completeness of reaction.

In one aspect is provided a method of producing an inclusion complex comprising a sildenafil salt (e.g., sildenafil citrate) and alpha-cyclodextrin by admixing the sildenafil salt with the alpha-cyclodextrin. In some embodiments, the method further comprises adding a solvent, mixed solvent, or buffer to the sildenafil salt, alpha-cyclodextrin, and/or mixture thereof.

In the preparation of the inclusion complex (e.g., an inclusion complex comprising alpha-cyclodextrin and sildenafil citrate) the suitable amount of solvent, mixed solvent, or buffer may be added directly to a solid mixture of the sildenafil salt and alpha-cyclodextrin. Alternatively, the solvent, mixed solvent, or buffer may be added to either the sildenafil salt or the alpha-cyclodextrin, and then added to the other of the sildenafil salt or the alpha-cyclodextrin. In some embodiments, the solvent, mixed solvent, or buffer may be added independently to each of the sildenafil salt and alpha-cyclodextrin, followed by combining the sildenafil salt and alpha-cyclodextrin.

In one aspect, the method of producing an inclusion complex of a sildenafil salt (e.g., sildenafil citrate) and alpha-cyclodextrin comprises the steps of: (a) admixing the sildenafil salt and alpha-cyclodextrin; and (b) adding a suitable amount of solvent, mixed solvent, or buffer to the mixture of step (a) and mixing until a suspension or solution is formed.

In some embodiments, the solvent, mixed solvent, or buffer is a buffer. Suitable buffers include, without limitation, phosphate buffers (e.g., phosphate-citrate), potassium hydrogen phthalate buffers, and acetate buffers. In some embodiments, the buffer is a phosphate-citrate buffer. In some embodiments, the added buffer and/or resulting suspension or solution has a pH between about 1.0, 2.0, or 3.0 and about 6.0, 7.0 or 8.0; about 3.0 and about 7.0, about 4.0 and about 6.0, about 4.5 and about 5.5; or a pH of greater than, less than, or about any of 1.0, 2.0, 3.0, 3.5, 4.0, 4.5, 5.0, 5.5, 6.0, 7.0, 7.5, or 8.0. In some embodiments, the solvent, mixed solvent, or buffer is a solvent, such as an organic solvent or water. Suitable organic solvents are known to those of skill in the art and include, but are not limited to, THF, methylene chloride, diethyl ether, petroleum ether, ethyl acetate, dioxane, DMF, DMSO, acetone, acetonitrile, ethanol, methanol, and pyridine. In some embodiments, the solvent is a polar solvent, such as water (e.g., ddH₂O), methanol, ethanol, DMSO, DMF, and pyridine. In some embodiments, the solvent, mixed solvent, or buffer is a mixed solvent, such as a mixture of water and an organic solvent. Suitable solvents, mixed solvents, or buffers include 100% of ddH₂O, or ddH₂O or buffer together with ethanol or methanol (1-99%).

For the methods of producing an inclusion complex of a sildenafil salt (e.g., sildenafil citrate) and alpha-cyclodextrin, the sildenafil salt may be admixed with the alpha-cyclodextrin at a molar ratio from about 0.2:1 to 50:1. In some embodiments, the molar ratio is about 0.5:1 to 1:40, or about 1:1 to 1:25, or about 1:1 to 1:20, or about 1:1 to 1:15, or about 1:1 to 1:10, or about 1:1 to 1:5, or about 1:1 to 1:3, or about 1:1 to 1:2, inclusive, or any of about 1:1, 1:2, 1:3, 1:4, 1:5, 1:6, or 1:7.

In some embodiments of the methods of producing an inclusion complex, the solvent, mixed solvent, or buffer is heated to less than, greater than, or about any of 25° C., 30° C., 35° C., 40° C., 45° C., 50° C., 55° C., 60° C., 65° C., 75° C., or 80° C. (e.g., before, during and/or after mixing). The solvent, mixed solvent, or buffer may be heated prior to and/or after being added to the sildenafil salt and/or alpha-cyclodextrin. In some embodiments, the solvent, mixed solvent, or buffer is heated greater than the preferred temperature for less than, greater than, or about any of 0.1 hr, 0.2 hr, 0.3 hr, 0.5 hr, 0.75 hr, 1 hr, 2 hr, 3 hr, 4 hr, 5 hr, 7 hr, 10 hr, 15 hr, 24 hr, 36 hr, or 48 hr.

During the formation of the inclusion complex between the sildenafil salt and alpha-cyclodextrin, a suitable polymer may be added which may enhance the solubility and/or complexation ability of the sildenafil salt and alpha-cyclodextrin inclusion complex. Accordingly, in some embodiments of the methods of producing an inclusion complex, step (a) further comprises admixing a suitable polymer. Suitable polymers include, for example, polyvinylpyrrolidone, hydroxypropyl methylcellulose, carboxymethylcellulose, and Plasdone® Povidone, and derivatives thereof. In some embodiments, the suitable polymer is a water-soluble polymer.

In some embodiments of the methods of producing an inclusion complex, the mixing is continued for at least any of about 0.1 hr, 0.2 hr, 0.3 hr, 0.5 hr, 0.75 hr, 1 hr, 2 hr, 4 hr, 10 hr, 24 hr, 36 hr, or 48 hr following formation of the suspension or solution. If heat is applied to the solvent, mixed solvent, or buffer during a method of producing an inclusion complex, the described mixing of the may occur prior to, simultaneously with, and/or after the application of said heat.

In some embodiments, the method of producing an inclusion complex further comprises a step for drying the product of step (b). In some embodiments, the drying comprises evaporation. In some embodiments, the evaporation occurs for greater than, less than, or about any of 0.1 hr, 0.25 hr, 0.5 hr, 1 hr, 2 hr, 5 hr, 10 hr, 1 day, or 5 days. In some embodiments, the evaporation is conducted under vacuum (e.g., less than any of about 0.75 atm, 0.5 atm, or 0.25 atm). In some embodiments, the evaporation is conducted under atmospheric pressure. In some embodiments, the drying comprises spray-drying. In some embodiments, the drying comprises freeze-drying. In some embodiments, the drying comprises spray-granulation.

In one aspect, is provided a method of producing an inclusion complex between a sildenafil salt (e.g., sildenafil citrate) and alpha-cyclodextrin comprising the steps of: (a) mixing the appropriate amount of sildenafil salt and alpha-cyclodextrin with or without a suitable polymer; (b) adding a suitable amount of solvent, mixed solvent, and/or buffer to the mixture of step (a) with vigorous mixing until a paste or a slurry is formed; (c) continuing the mixing with further addition of solvent (e.g., water), mixed solvent, or buffer if necessary to maintain the paste or the slurry consistency, for a suitable period of time to form the inclusion complex; and (d) drying the product of step (c). In some embodiments of step (b), the buffer is a phosphate-citrate buffer and the pH is about 5. In some embodiments, the solvent added during steps (b) and (c) is heated. In some embodiments, wherein the solvent, mixed solvent, or buffer is deionized water and/or a buffer, the deionized water and/or a buffer is heated to about 60° C. In some embodiments, the mixing is preferably continued for a period of time greater than 0.2 hours. In some embodiments, the vigorous mixing until a paste or a slurry is formed is conducted at about 60° C.

In another aspect, is provided a method of producing an inclusion complex between a sildenafil salt (e.g., sildenafil citrate) and alpha-cyclodextrin comprising the steps of: (a) mixing suitable amounts of sildenafil salt and alpha-cyclodextrin with or without a suitable polymer; (b) adding of a suitable solvent, mixed solvent, and/or buffer to the mixture of step (a) with mixing until a slurry, suspension or solution is formed; and (c) allowing the formation of the inclusion complex by evaporation of the water over a period of time. In some embodiments of step (b), the buffer is a phosphate-citrate buffer and the buffer pH is about 5. In some embodiments, the solvent added during step (b) is heated. In some embodiments, wherein the solvent, mixed solvent, or buffer is deionized water and/or a buffer, the deionized water and/or a buffer is heated to about 60° C. In some embodiments of step (c), heat is applied to increase the evaporation rate. In some embodiments, the evaporation is conducted at 40° C. In some embodiment, evaporation in step (c) occurs for greater than about 1 hour. In some embodiments, the evaporation is conducted under vacuum.

In another aspect, is provided a method of producing an inclusion complex between a sildenafil salt (e.g., sildenafil citrate) and alpha-cyclodextrin comprising the steps of: (a) mixing suitable amounts of sildenafil salt and alpha-cyclodextrin with or without a suitable polymer; (b) adding of a suitable solvent, mixed solvent, and/or buffer to the mixture of step (a) with mixing until a slurry, suspension or solution is formed; and (c) spray-drying the slurry, suspension or solution to obtain a solid drug-cyclodextrin inclusion complex. In some embodiments, the buffer is a phosphate-citrate and the buffer pH is about 5. In some embodiments, the suitable solvent, mixed solvent, and/or buffer added during step (b) it heated. In some embodiments, wherein the solvent, mixed solvent, or buffer is deionized water and/or a buffer, the deionized water and/or a buffer is heated to about 60° C.

In another aspect, is provided a method of producing an inclusion complex between a sildenafil salt (e.g., sildenafil citrate) and alpha-cyclodextrin comprising the steps of: (a) mixing suitable amounts of sildenafil salt and alpha-cyclodextrin with or without a suitable polymer; (b) adding of a suitable solvent, mixed solvent, and/or buffer to the mixture of step (a) with mixing until a solution is formed; and (c) freeze-drying the solution to obtain a solid drug-cyclodextrin inclusion complex. In some embodiments, the buffer is a phosphate-citrate buffer and the buffer pH is about 5. In some embodiments, the solvent, mixed solvent, and/or buffer added during step (b) is heated. In some embodiments, wherein the solvent, mixed solvent, or buffer is deionized water and/or a buffer, the deionized water and/or a buffer is heated to about 60° C.

In another aspect, is provided a method of producing an inclusion complex between a sildenafil salt (e.g., sildenafil citrate) and alpha-cyclodextrin comprising the steps of: (a) mixing suitable amounts of sildenafil salt and alpha-cyclodextrin with or without a suitable polymer; (b) adding of a suitable solvent, mixed solvent, and/or buffer to the mixture of step (a) with mixing until a slurry, suspension or solution is formed; (c) adding inactive pharmaceutical excipients to the slurry, suspension or solution, with continued mixing and (d) spray-granulating the slurry, suspension or solution to obtain solid particles, suitable for formulation into an oral formulation, containing a solid drug-cyclodextrin inclusion complex. In some embodiments, the buffer is a phosphate-citrate buffer and the buffer pH is about 5. In some embodiments, the solvent, mixed solvent, or buffer added during step (b) may be heated. In some embodiments, wherein the solvent, mixed solvent, or buffer is deionized water and/or a buffer, the deionized water and/or a buffer is heated to about 60° C. The inactive pharmaceutical excipients included to produce an oral formulation according to step (c) may include commonly used pharmaceutical excipients commonly used in the art, and/or those described herein.

In another aspect, is provided a method of producing an inclusion complex between a sildenafil salt (e.g., sildenafil citrate) and alpha-cyclodextrin comprising the steps of: (a) mixing suitable amounts of sildenafil salt and alpha-cyclodextrin with or without a suitable polymer; (b) adding a suitable solvent, mixed solvent, and/or buffer to the mixture of step (a) with mixing until a solution is formed; (c) producing a liquid oral formulation, by the addition of inactive pharmaceutical excipients to the solution, containing the liquid drug-cyclodextrin inclusion complex. In some embodiments, the buffer is a phosphate-citrate buffer and the buffer pH is about 5. In some embodiments, the solvent, mixed solvent, and/or buffer added during step (d) is heated. In some embodiments, wherein the solvent, mixed solvent, or buffer is deionized water and/or a buffer, the deionized water and/or a buffer is heated to about 60° C. The inactive pharmaceutical excipients included to produce a liquid oral formulation according to step (c) may include commonly used pharmaceutical excipients commonly used in the art, and/or those described herein.

In some embodiments of the methods for producing an inclusion complex between a sildenafil salt (e.g., sildenafil citrate) and alpha-cyclodextrin described above, greater than any of about 10%, 15%, 20%, 30%, 40%, 50%, 60%, 70%, 80%, 85%, 90%, 95%, 97%, 98%, or 99% of the sildenafil salt is in complexed form.

### Formulations

The inclusion complexes described herein (e.g., an inclusion complex of sildenafil citrate with alpha-cyclodextrin) may be used in the preparation of a formulation, such as a pharmaceutical composition or formulation, by combining an inclusion complex described with a pharmaceutical acceptable carrier, excipients, stabilizing agents and/or other agents, which are known in the art, for use in the methods of treatment, methods of administration, and dosage regimes described herein. The formulations may vary or be tailored according to the condition to be treated, the amount of compound to be administered, the condition of the individual, and other variables that will readily be apparent to one of ordinary skill in the art in view of the teachings provided herein. The inclusion complexes may be formulated, for example, as solid, semi-solid, and liquid dosage forms, such as tablets, pills, powders, liquid solutions or suspensions, suppositories, injectable and infusible solutions, and sprays. The preferred form depends on the intended mode of administration and therapeutic application. The following formulations, additives, and methods are merely exemplary and are in no way limiting.

The sildenafil salts described herein (e.g., sildenafil citrate) may be formulated with alpha-cyclodextrin and may comprise one or more of the favorable properties described for the inclusion complexes herein (e.g., increased solubility). In some embodiments, the sildenafil salt is in the uncomplexed form in presence of alpha-cyclodextrin. In some embodiments, is provided a mixture of sildenafil salt in both complexed and uncomplexed form with alpha-cyclodextrin (e.g., a molar ratio mixture of greater than, less than, or any of about 1:100, 1:50, 1:25, 1:15, 1:10, 1:7.5, 1:5, 1:3, 1:2, 1:1, 2:1, 3:1, 4:1, 5:1, 7.5:1, 10:1, 15:1, 25:1, 50:1, or 100:1 of complexed sildenafil salt to uncomplexed sildenafil salt, respectively). Accordingly, in one aspect is provided a formulation comprising a sildenafil salt, alpha-cyclodextrin, and a carrier. In some embodiments, the formulation comprises an effective amount a sildenafil salt, alpha-cyclodextrin, and a carrier. The formulation may comprise a molar ratio of alpha-cyclodextrin to sildenafil salt that is greater than, less than, or any of about 1:1, 2:1, 3:1, 4:1, 5:1, 7.5:1, 10:1, 15:1, 25:1, 50:1, or 100:1. Additionally, the formulation comprising the sildenafil salt, alpha-cyclodextrin, and a carrier may be further formulated in any manner described below for the inclusion complex formulations, and may be used in any of the methods described herein, as well as at any dosage described herein, for the inclusion complexes and/or inclusion complex formulations (e.g., to treat a condition, such as sexual dysfunction or pulmonary hypertension). These formulations also may provide improved pharmacokinetic properties as described herein (e.g., bioavailability, Cₘₐₓ, Tₘₐₓ, and time of onset) when compared to sildenafil salt (e.g., sildenafil citrate) administered under the same conditions.

In some embodiments, the formulation comprising the sildenafil salt and alpha-cyclodextrin, a complex of the sildenafil salt with alpha-cyclodextrin, or a mixture thereof, is a sterile formulation.

Additives used with the inclusion complexes described herein (e.g., an inclusion complex of sildenafil citrate with alpha-cyclodextrin) include, for example, one or more excipients (e.g., one or more excipients), antioxidants (e.g., one or more antioxidants), stabilizers (e.g., one or more stabilizers), preservatives (e.g., one or more preservatives), pH adjusting and buffering agents (e.g., one or more pH adjusting and/or buffering agents), tonicity adjusting agents (e.g., one or more tonicity adjusting agents), thickening agents (e.g., one or more thickening agents), suspending agents (e.g., one or more suspending agents), binding agents (e.g., one or more binding agents, viscosity-increasing agents (e.g., one or more viscosity-increasing agents), and the like, either alone or together with one or more additional pharmaceutical agents, provided that the additional components are pharmaceutically acceptable. In some embodiments, the formulation may include combinations of two or more of the additional components as described herein (e.g., any of 2, 3, 4, 5, 6, 7, 8, or more additional components). In some embodiments, the additives include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in REMINGTON'S PHARMACEUTICAL SCIENCES, Marck Pub. Co., New Jersey 18^{th} edition (1996), and REMINGTON: THE SCIENCE AND PRACTICE OF PHARMACY, Lippincott Williams & Wilkins, Philadelphia, 20^{th} edition (2003) and 21^{st} edition (2005).

Formulations suitable for oral administration may comprise, for example, (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art. Oral formulations may include any suitable dosage, including those described herein, such as any of about 0.1 mg, 0.25 mg, 0.5 mg, 0.75 mg, 1 mg, 1.5 mg, 2.5 mg, 5 mg, 10 mg, 20 mg, 25 mg, 50 mg, 75 mg, or 100 mg of sildenafil within the complex (as measured from the amount of sildenafil base or sildenafil salt). As described below, the formulations may be used for the treatment of a condition (e.g., sexual dysfunction or pulmonary hypertension). Solid dosage forms for oral administration may be particularly useful for the treatment of erectile dysfunction.

The inclusion complexes can be enclosed in a hard or soft capsule, can be compressed into tablets, or can be incorporated with beverages or food or otherwise incorporated into the diet. Capsules can be formulated by mixing the inclusion complex with an inert pharmaceutical diluent and inserting the mixture into a hard gelatin capsule of the appropriate size. If soft capsules are desired, a slurry of the inclusion complex with an acceptable vegetable oil, light petroleum or other inert oil can be encapsulated by machine into a gelatin capsule.

Liquid dosage forms for oral administration may include pharmaceutically acceptable emulsions, solutions, suspensions, syrups, and elixirs containing inert diluents commonly used in the art, such as water. Such formulations may also comprise adjuvants, such as wetting agents, emulsifying and suspending agents, cyclodextrins, and sweetening, flavoring, and perfuming agents. Liquid dosage forms of the inclusion complexes for oral administration may be particularly useful for the treatment of pulmonary hypertension (e.g., in a neonate).

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a freeze-dried (lyophilized) condition requiring only the addition of the sterile liquid excipient methods of treatment, methods of administration, and dosage regimes described herein (i.e., water) for injection, immediately prior to use. Extemporaneous injection solutions and suspensions can be prepared from sterile powders, granules, and tablets of the kind previously described.

Injectable preparations (for example, sterile injectable aqueous or oleaginous suspensions) may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. The sterile injectable preparation may also be a sterile powder to be reconstituted using acceptable vehicles prior to administration. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the preparation of injectables.

Formulation of the inclusion complex in liquid form (for oral administration, parenteral administration, or otherwise) may have a pH in the range of about 4.5 to about 9.0, including for example pH ranges of any of about 5.0 to about 8.0, about 5.0 to about 7.0, about 5.0 to about 6.0, about 6.5 to about 7.5, and about 6.5 to about 7.0. In some embodiments, the pH of the composition is formulated to no less than about 6, 5, or 4, including for example a pH of any of about 8, 7.5, 7, 6.5, 6, 5.5, 5, 4.5, or 4. The formulation can also be made to be isotonic with blood by the addition of a suitable tonicity modifier, such as glycerol.

The inclusion complexes may also be formulated for administration by inhalation. Formulations suitable for aerosol administration which comprise the inclusion complex may include, for example, aqueous and non-aqueous, isotonic sterile solutions, which can contain anti-oxidants, buffers, bacteriostats, and solutes, as well as aqueous and non-aqueous sterile suspensions that can include suspending agents, solubilizers, thickening agents, stabilizing agents, and preservatives, alone or in combination with other suitable components, which can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

The inclusion complexes may also be formulated in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The inclusion complexes may also be formulated for topical administration, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

Also provided are unit dosage forms comprising the formulations described herein. These unit dosage forms can be stored in a suitable packaging in single or multiple unit dosages and may also be further sterilized and sealed. For example, the pharmaceutical formulation (e.g., a dosage or unit dosage form of a pharmaceutical formulation) may include (i) a mixture of a sildenafil salt (e.g., sildenafil citrate) with alpha-cyclodextrin, and/or an inclusion complex thereof, and (ii) a pharmaceutically acceptable carrier. In various variations, the amount of sildenafil (as measured from the amount of sildenafil base or sildenafil salt) within the formulation is included in any of the following ranges: about 0.1 to about 50 mg, about 1 to about 50 mg, about 5 to about 50 mg, about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of sildenafil base or sildenafil salt within the formulation (e.g., a dosage or unit dosage form) is in the range of about 1 mg to about 500 mg, such as any of about 5 mg to about 250 mg, about 20 mg to about 100 mg, or about 20 mg, 25 mg, 50 mg, 75 mg, or 100 mg. In some of these embodiments, the formulation may comprise a molar ratio of alpha-cyclodextrin to sildenafil salt is that is greater than, less than, or any of about 1:1, 2:1, 3:1, 4:1, 5:1, 7.5:1, 10:1, 15:1, 25:1, 50:1, or 100:1. For example, in some embodiments wherein the amount of the amount of sildenafil base or sildenafil salt within the formulation is in the range of about 1 mg to about 500 mg, such as any of about 5 mg to about 250 mg, 20 mg to about 100 mg, or about 20 mg, 25 mg, 50 mg, 75 mg, or 100 mg, the amount of alpha-cyclodextrin in the formulation may be any of about 5 mg to about 1500 mg, such as any of about 15 mg to about 1000 mg, about 30 mg to about 750 mg, about 60 mg to about 300 mg, about 100 mg to about 200 mg, or about 60 mg, 75 mg, 100 mg, 125 mg, 150 mg, 200 mg, 225 mg, 250 mg, 275 mg or 300 mg. In some embodiments, the carrier is suitable for parental administration (e.g., intravenous administration). In some embodiments, the carrier is suitable for oral administration. In some embodiments, the sildenafil of the inclusion complex is the only pharmaceutically active agent for the treatment of the condition (e.g., erectile dysfunction or pulmonary hypertension) that is contained in the formulation.

In some embodiments, are provided dosage forms (e.g., a unit dosage form) for the treatment of erectile dysfunction or pulmonary hypertension, comprising (i) a mixture of a sildenafil salt (e.g., sildenafil citrate) with alpha-cyclodextrin, and/or an inclusion complex thereof; and alpha-cyclodextrin, wherein the amount of sildenafil (as measured from the amount of sildenafil base or sildenafil salt) is in the range of about 0.1 mg to about 100 mg, and (ii) a pharmaceutically acceptable carrier. In some embodiments, the amount of sildenafil in the unit dosage form includes any of about 0.1 mg, 1 mg, 2.5 mg, 5 mg, 10 mg, 20 mg, 25 mg, 50 mg, or 100 mg of sildenafil.

In some embodiments, are provided formulations of the inclusion complexes described herein comprising one or more of a complexing agent, a filler, a diluent, a granulating agent, a disintegrant, a lubricant, or a glidant. The complexing agent, filler, diluent, granulating agent, disintegrant, lubricant, or glidant may be chosen from among the ingredients listed in Table 1. In some embodiments, formulations may contain zero, one, or more than one ingredient from each use category in Table 1. Formulations may additionally contain other complexing agents, fillers, diluents, granulating agents, disintegrants, lubricants, or glidants not listed in Table 1. Formulations may also contain additional ingredients that are not complexing agents, fillers, diluents, granulating agents, disintegrants, lubricants, or glidants.

TABLE 1

| Use | Ingredients |
|---|---|
| Complexing agent | Sodium hydrogen carbonate; ethanol; methanol |
| Filler/diluent | Microcrystalline cellulose; calcium carbonate; glucose; calcium |
| | hydrogen phosphate; lactose; mannitol; sodium chloride; sucrose; |
| | dextrates; microfine cellulose; modified starch; sucrose-dextrin co- |
| | precipitate |
| Granulating agent | Acacia mucilage; glucose; gelatine; povidone (PVP); starch |
| | mucilage; sucrose; tragacanth mucilage |
| Disintegrant | Sodium hydroxymethylcellulose; alginic acid; sodium alginate; |
| | aluminium magnesium silicate; carbon dioxide; carmellose sodium; |
| | cationic exchange resins; croscarmellose sodium; microcrystalline |
| | cellulose; modified starch; |
| | sodium lauryl sulphate; sodium glycine carbonate; sodium starch |
| | glycollate; |
| | starch |
| Lubricant | Magnesium stearate; calcium stearate; stearic acid; fumaric acid; |
| | hydrogenated vegetable oil; liquid paraffin; magnesium lauryl |
| | sulphate; |
| | macrogol 4000 and 6000; sodium benzoate; sodium lauryl sulphate; |
| | sodium stearyl fumarate |
| Glidant | Colloidal silica; talc |

Exemplary formulations are shown in Table 2.

TABLE 2

| | Formulation | Formulation | Formulation | Formulation | Formulation |
|---|---|---|---|---|---|
| | 1 | 2 | 3 | 4 | 5 |
| Complexing | Sodium | Ethanol | None | Methanol | None |
| agent | hydrogen | | | | |
| | carbonate | | | | |
| Filler/diluent | Glucose | Mannitol; | Microcrystalline | Sodium | Glucose; |
| | | modified | cellulose; | chloride; | sucrose |
| | | starch | lactose | dextrates | |
| Granulating | None | Acacia | None | Starch mucilage | Tragacanth |
| agent | | mucilage | | | mucilage |
| Disintegrant | Alginic acid; | Aluminum | Crosscarmellose | Microcrystalline | Sodium lauryl |
| | carbon dioxide | magnesium | sodium | cellulose | sulphate |
| | | silicate | | | |
| Lubricant | Calcium | Fumaric acid | Magnesium | Liquid paraffin | Sodium |
| | stearate | | stearate | | benzoate |
| Glidant | Colloidal | Talc | Colloidal | Talc | Talc |
| | silica | | silica | | |
| Other | Dye | Dye | Dye; PVA- | PVA-based film | None |
| ingredients | | | based film | coating | |
| | | | coating | | |

In some embodiments, the complexing agent, filler, diluent, granulating agent, disintegrant, lubricant, or glidant is present in the amount per tablet indicated in Table 3.

TABLE 3

| Ingredient | Amount per tablet |
|---|---|
| Complexing agent | 1-200 mg |
| Filler/diluent | 100-200 mg |
| Granulating agent | 1-50 mg |
| Disintegrant | 1-50 mg |
| Lubricant | 1-10 mg |
| Glidant | 5-30 mg |

In some embodiments, the inclusion complex is formulated as a tablet comprising sildenafil citrate and alpha-cyclodextrin in amounts per tablet as indicated in Table 4 and one or more additional ingredients listed in Table 4 in an amount per tablet as indicated in Table 4.

TABLE 4

| Ingredient | Amount Per Tablet |
|---|---|
| Sildenafil citrate | 60, 61, 62, 63, 64, 65, 66, 67, 68, 69, 70, |
| | 71, 72, 73, 74, 75, 76, 77, 78, 78, or |
| | 80 mg |
| Alpha-cyclodextrin | 150, 160, 170, 180, 190, 200, 210, 220, |
| | 230, 240, or 250 mg |
| Microcrystalline cellulose, | 150, 160, 165, 170, 180, 185, 190, 195, or |
| glucose, mannitol, or sucrose | 200 mg |
| Aluminum magnesium | 5, 10, 15, 20, 25, 30, 35, 40, 45, 50, 55, |
| silicate, carmellose sodium, | or 60 mg |
| crosscarmellose, or modified | |
| starch | |
| Colloidal silica or talc | 1, 2, 3, 4, 5, 6, 7, 8, 9, 10, 11, 12, 13, 14, |
| | 15, 16, 17, 18, 19, or 20 mg |
| Dextrates, lactose, or sodium | 0.5, 1.0, 1.5, 2.0, 2.5, 3.0, 3.5, 4.0, 4.5, or |
| chloride | 5.0 mg |
| Dye | 0.5, 1.0, 1.5, 2.0, 2.5, 3.0, 3.5, 4.0, 4.5, or |
| | 5.0 mg |
| Polymer-based film coating | 1, 2, 3, 4, 5, 6, 7, 8, 9, 10, 11, 12, 13, 14, |
| | 15, 16, 17, 18, 19, or 20 mg |

### Kits

Also provided are kits containing materials useful for the treatment of a condition that is responsive to the sildenafil (e.g., sexual dysfunction or pulmonary hypertension). The kits may contain an inclusion complex of a sildenafil salt (e.g., sildenafil citrate) and alpha-cyclodextrin, and optionally contain instructions for use (e.g., instructions for preparation and/or administration of a formulation comprising the complex). Information detailing possible side effects of the formulation, and any other relevant information may also be enclosed. The instructions may be in any suitable format, including, but not limited to, printed matter, videotape, computer readable disk, optical disc or directions to internet-based instructions.

In one aspect, is provided a kit for treating an individual who suffers from or is susceptible to one or more of the conditions described herein, comprising a first container comprising a dosage amount of a formulation containing an inclusion complex as disclosed herein, and instructions for use. The container may be any of those known in the art and appropriate for storage and delivery of intravenous formulation. In certain embodiments the kit further comprises a second container comprising a pharmaceutically acceptable carrier, diluent, adjuvant, etc. for preparation of the formulation to be administered to the individual.

In some embodiments, the kits comprise a container with a label. Suitable containers include, for example, bottles, vials, and test tubes. The containers may be formed from a variety of materials such as glass or plastic. The containers may hold an inclusion complex comprising a sildenafil salt and alpha-cyclodextrin (e.g., a formulation the complex and further comprising one or more additional pharmaceutical agents). The label on the container may indicate that the inclusion complex or the formulation is used for treating or suppressing a condition that is responsive to sildenafil (e.g., sexual dysfunction or pulmonary hypertension), and may also indicate directions for either in vivo or in vitro use, such as those described herein.

The kit may further include other materials desirable from a commercial and user standpoint, including other buffers, diluents, filters, needles, syringes, and package inserts with instructions for performing any methods described herein. In some embodiments, the kit comprises the container described above and a second container comprising a buffer.

The kits may include additional pharmaceutical agents for use in conjunction with the formulation described herein. In some variations, the additional pharmaceutical agent(s) may be one or more drug(s) for the treatment of conditions responsive to sildenafil (e.g., sexual dysfunction or pulmonary hypertension). In some variations, the additional pharmaceutical agent(s) may be one or more drug(s) for the treatment of one or more side effects from the use of the inclusion complexes described herein. These agents may be provided in a separate form, or mixed with the complexes described herein, provided such mixing does not reduce the effectiveness of either the pharmaceutical agent or formulation described herein and is compatible with the route of administration. Similarly the kits may include additional agents for adjunctive therapy or other agents known to the skilled artisan as effective in the treatment or prevention of the conditions described herein.

Kits may also be provided that contain sufficient dosages of the compounds described herein (including formulations thereof) to provide effective treatment for an individual for an extended period, such as any of 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

The kits may include the composition as described herein packaged in either a unit dosage form or in a multi-use form. The kits may also include multiple units of the unit dose form. The kits may be used for any of the methods described herein, including, for example, to treat an individual with a condition described herein, or to delay a condition described herein. In certain embodiments the kits may include a dosage amount of at least one formulation as disclosed herein. Kits may also comprise a means for the delivery of the formulation thereof.

### Methods of Use

The inclusion complexes described herein comprising a sildenafil salt and alpha-cyclodextrin, formulations thereof, and formulations comprising an uncomplexed sildenafil salt and alpha-cyclodextrin, or mixtures thereof, may be used to treat conditions responsive to sildenafil and/or interact with an enzyme target responsive to sildenafil.

For example, an inclusion complex comprising a sildenafil salt and alpha-cyclodextrin may be used to inhibit the cyclic guanosine monophosphate (cGMP)-specific phosphodiesterase 12 type-5 (PDE5) enzyme. Accordingly, in one embodiment is comprised a method of inhibiting a PDE5 enzyme, comprising contacting the PDE5 enzyme with an effective amount of the sildenafil salt (e.g., sildenafil citrate) of the inclusion complex comprising the sildenafil salt and alpha-cyclodextrin. In some embodiments, the inclusion complex comprising a sildenafil salt and alpha-cyclodextrin is capable of selectively inhibiting the PDE5 enzyme over other cyclic guanosine monophosphate (cGMP)-specific phosphodiesterase enzymes (e.g., PDE1, PDE3, PDE4, PDE6, PDE7, PDE8, PDE9, PDE10 and PDE11). In some of these embodiments, the IC₅₀ for PDE5 is more than any of about 2-, 5-, 10-, 20-, 50-, 100-, 200-, 500-, 1000-, 2000-, or 5000-fold lower than the IC₅₀ of the other cyclic guanosine monophosphate (cGMP)-specific phosphodiesterase enzyme.

The inclusion complexes described herein may be useful in the treatment of conditions that may be related to the PDE5 enzyme. For example, an inclusion complex comprising a sildenafil salt (e.g., sildenafil citrate) and alpha-cyclodextrin may be used for the treatment of conditions such as sexual dysfunction (e.g., erectile dysfunction and female sexual arousal disorder), circulatory disorders (e.g., pulmonary hypertension), cystic fibrosis, achalasia, subarachnoid hemorrhage, cerebral ischemia, platelet aggregation, cardioprotection, memory retention, diabetes, Raynaud's Phenomenon and altitude sickness.

In one aspect, the inclusion complexes described herein (e.g., an inclusion complex comprising sildenafil citrate and alpha-cyclodextrin) may be used for the treatment sexual dysfunction (e.g., erectile dysfunction and female sexual arousal disorder). In one embodiment is provided a method of treating sexual dysfunction (e.g., erectile dysfunction) in an individual, comprising administering to the individual an effective amount of an inclusion complex comprising a sildenafil salt (e.g., sildenafil citrate) and alpha-cyclodextrin, or a formulation thereof.

In another aspect, the inclusion complexes described herein (e.g., an inclusion complex comprising sildenafil citrate and alpha-cyclodextrin) may be used for the treatment of pulmonary hypertension (e.g., arterial, venous, hypoxic, thromboembolic, and/or miscellaneous pulmonary hypertension). In one embodiment is provided a method of treating pulmonary hypertension in an individual, comprising administering to the individual an effective amount of an inclusion complex comprising a sildenafil salt (e.g., sildenafil citrate) and alpha-cyclodextrin, or a formulation thereof. In some embodiments, the pulmonary hypertension is neonatal pulmonary hypertension and the individual is a neonate. As described below, the inclusion complex may be administered via any route (e.g., orally or parenterally, such as intravenously).

In some variations, the individual being treated for a condition described herein (e.g., sexual dysfunction or pulmonary hypertension) has been identified as having one or more of the symptoms described herein. Identification of the conditions as described herein by a skilled physician is routine in the art such as routine physical exams or clinical detection (e.g., duplex ultrasound, penile nerves function, nocturnal penile tumescence (NPT), penile biothesiometry, penile angiogram, dynamic infusion cavernosometry, corpus cavernosometry, digital subtraction angiography (DSA), magnetic resonance angiography (MRA) for detection of erectile dysfunction; and pulmonary function tests, electrocardiography (ECG), arterial blood gas measurements, X-rays of the chest, and high-resolution CT scanning, decreased pulmonary artery occlusion pressure (PAOP or PCWP), and increased pulmonary vascular resistance (PVR) for detection of pulmonary hypertension) and may also be suspected by the individual or others, for example, due to the inability to obtain or maintain an erection, inability to obtain or maintain a nocturnal erection, obtaining erections which are either not rigid or full (lazy erection), or an erection lost more rapidly than would be expected for erectile dysfunction; and shortness of breath, fatigue, cough, angina pectoris, fainting or syncope, peripheral edema, hemoptysis for pulmonary hypertension. In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits, and/or by the diagnosis of other factors which may be associated and/or lead to the condition (e.g., spinal-cord injury, depression, diabetes, atherosclerosis, hypertension, ischemic heart disease, or hypogonadism for erectile dysfunction; and cirrhosis, collagen vascular disease (e.g. scleroderma), portal hypertension, HIV, venous or capillary disease, atrial or ventricular disease, valvular disease (e.g. mitral stenosis), emphysema, hypoxemia, chronic obstructive pulmonary disease (COPD), interstitial lung disease (ILD), sleep-disordered breathing, alveolar hypoventilation, chronic thrombotic and embolic disease, for pulmonary hypertension).

In some embodiments, the methods and/or inclusion complex formulations used herein reduce the severity of one or more symptoms associated with the condition (e.g., erectile dysfunction or pulmonary hypertension) by at least any of about 10%, 20%, 30%, 40%, 50%, 60%, 70%, 80%, 90%, 95%, or 100% compared to the corresponding symptom in the same individual prior to treatment or compared to the corresponding symptom in other individuals not receiving the methods and/or inclusion complex formulations.

### Dosing and Methods of Administration

The amount of the inclusion complex administered to an individual (such as a human) and/or the amount administered in order to achieve an effective amount may vary based on variety of factors, including, for example, the particular condition being treated, the frequency of administration, the particular formulation being administered, the severity of the condition being treated and the age, weight and general health of the individual, the adverse effects experienced by the individual being treated, etc. A pharmaceutical unit dosage chosen may be fabricated and administered to provide a defined final concentration of drug in the blood, tissues, organs, or other targeted region of the body. Determination of an effective amount for a given situation can be readily determined by routine experimentation (e.g., using in vivo animal models) and is within the skill and judgment of the ordinary clinician, particularly in view of the teachings provided herein.

In some embodiments, the amount of the inclusion complex comprising a sildenafil salt (e.g., sildenafil citrate) and alpha-cyclodextrin is effective to result in an objective response (such as a partial response or a complete response). In some embodiments, the amount of inclusion complex is sufficient to result in a complete response in the individual. In some embodiments, the amount of the inclusion complex is sufficient to result in a partial response in the individual. In some embodiments, the amount of the inclusion complex administered alone is sufficient to produce an overall response rate of more than about any of about 10%, 20%, 30%, 40%, 50%, 60%, 70%, 80%, 90%, or 95% among a population of individuals treated with the complex. Responses of an individual to treatment can be determined by the skilled artisan using, for example, routine physical exams and/or clinical detection known in the art and/or described herein.

In some embodiments, the amount of the inclusion complex is below the level that induces a toxicological effect (i.e., an effect above a clinically acceptable level of toxicity) or is at a level where a potential side effect can be controlled or tolerated when the complex is administered to the individual. In some embodiments, the amount of the inclusion complex is close to a maximum tolerated dose (MTD) of the complex following the same dosing regime. In some embodiments, the amount of the inclusion complex is more than any of about 80%, 90%, 95%, or 98% of the MTD.

In some embodiments, the amount of sildenafil (as measured from the amount of sildenafil base or sildenafil salt) from an inclusion complex comprising the sildenafil salt (e.g., sildenafil citrate) and alpha-cyclodextrin is included in any of the following ranges: about 0.1 to about 5 mg, about 1 to about 10 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of sildenafil from an inclusion complex is in the range of about 1 mg to about 500 mg, such as about 5 mg to about 400 mg, 10 mg to about 300 mg, 20 mg to about 200 mg, or about 20 mg, 25 mg, 50 mg, 75 mg, or 100 mg. In some embodiments of the liquid formulations, the concentration of sildenafil (as measured from the amount of sildenafil base or sildenafil salt) from an inclusion complex comprising the sildenafil salt (e.g., sildenafil citrate) and alpha-cyclodextrin is inclusion complex is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the sildenafil of the inclusion complex is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary effective amounts of sildenafil (as measured from the amount of sildenafil base or sildenafil salt) from an inclusion complex comprising the sildenafil salt (e.g., sildenafil citrate) and alpha-cyclodextrin include, but are not limited to, any of about 25 mg/m², 30 mg/m², 50 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m². In various variations, a formulation includes less than about any of 350 mg/m², 300 mg/m², 250 mg/m², 200 mg/m², 150 mg/m², 120 mg/m², 100 mg/m², 90 mg/m², 50 mg/m², or 30 mg/m² of sildenafil. In some embodiments, the amount of sildenafil within the inclusion complex per administration is less than any of about 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of sildenafil from the inclusion complex is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m².

In some embodiments of any of the above aspects, the effective amount of sildenafil (as measured from the amount of sildenafil base or sildenafil salt) from an inclusion complex comprising the sildenafil salt (e.g., sildenafil citrate) and alpha-cyclodextrin includes at least any of about 0.1 mg/kg, 0.25 mg/kg, 0.5 mg/kg, 1 mg/kg, 1.5 mg/kg, 2 mg/kg, 2.5 mg/kg, 3.5 mg/kg, 5 mg/kg, 6.5 mg/kg, 7.5 mg/kg, 10 mg/kg, 15 mg/kg, or 20 mg/kg. In various variations, the effective amount of sildenafil includes less than any of about 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, 2 mg/kg, 1.5 mg/kg, 1 mg/kg, 0.75 mg/kg, 0.5 mg/kg, 0.25 mg/kg, 0.1 mg/kg, 0.05 mg/kg.

Exemplary dosing frequencies include, but are not limited to, weekly without break; weekly, three out of four weeks; once every three weeks; once every two weeks; weekly, two out of three weeks. In some embodiments, the inclusion complex is administered any of about once every 2 weeks, once every 3 weeks, once every 4 weeks, once every 6 weeks, or once every 8 weeks. In some embodiments, the composition is administered at least any of about 1×, 2×, 3×, 4×, 5×, 6×, or 7× (i.e., daily) a week. In some embodiments, the intervals between each administration are less than any of about 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than any of about 1 month, 2 months, 3 months, 4 months, 5 months, 6 months, 8 months, or 12 months. In some embodiments, there is no break in the dosing schedule. In some embodiments, the interval between each administration is no more than about a week. The administration of the inclusion complex can be extended over an extended period of time, such as from about a month up to about seven years. In some embodiments, the composition is administered over a period of at least any of about 2, 3, 4, 5, 6, 7, 8, 9, 10, 11, 12, 18, 24, 30, 36, 48, 60, 72, or 84 months. The dosing frequency of the inclusion complex may be adjusted over the course of the treatment based on the judgment of the administering physician.

The inclusion complexes described herein allow, in some embodiments, infusion of the complex to an individual over an infusion time that is shorter than about 24 hours. For example, in some embodiments, the inclusion complex is administered over an infusion period of less than any of about 24 hours, 12 hours, 8 hours, 5 hours, 3 hours, 2 hours, 1 hour, 30 minutes, 20 minutes, or 10 minutes. In some embodiments, the inclusion complex is administered over an infusion period of about 30 minutes.

Any of the inclusion complexes described herein (e.g., an inclusion complex comprising sildenafil citrate and alpha-cyclodextrin) can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In one variation, inclusion complex is administered by any acceptable route including, but not limited to, orally, intramuscularly, transdermally, intravenously, through an inhaler or other air borne delivery systems and the like. Additional methods of administration are known in the art.

In some embodiments, the inclusion complexes described herein (e.g., an inclusion complex comprising sildenafil citrate and alpha-cyclodextrin) are administered parenterally (e.g., intravenously). For example, in some embodiments are provided methods of treating pulmonary hypertension in an individual (e.g., an adult or a neonate) comprising parenterally (e.g., intravenously) administering an inclusion complex comprising a sildenafil salt and alpha-cyclodextrin.

The physiochemical properties (such as stability in vivo) of the inclusion complexes described herein (e.g., an inclusion complex comprising sildenafil citrate and alpha-cyclodextrin) may allow the complexes to be taken orally. In some embodiments, the inclusion complexes or formulations comprising the complexes are suitable for oral administration.

As described herein, the inclusion complexes may be administered with an additional therapeutic agent and/or an additional treatment modalitiy. The dosing frequency of the inclusion complex and the additional therapeutic agent may be adjusted over the course of the treatment based on the judgment of the administering physician. In some embodiments, the inclusion complex and the additional therapeutic agent are administered simultaneously, sequentially, or concurrently. When administered separately, the inclusion complex and the additional therapeutic agent can be administered at different dosing frequency or intervals. For example, the inclusion complex can be administered weekly, while the additional therapeutic agent can be administered more or less frequently. In some embodiments, sustained continuous release formulation of the inclusion complex and/or the additional therapeutic agent may be used. Various formulations and devices for achieving sustained release are known in the art. A combination of the administration configurations described herein can be used.

The present invention will be understood more readily by reference to the following examples, which are provided by way of illustration and are not intended to be limiting of the present invention.

### EXAMPLES

### Example 1

### Solubility of Sildenafil Citrate Complexed with Cyclodextrin

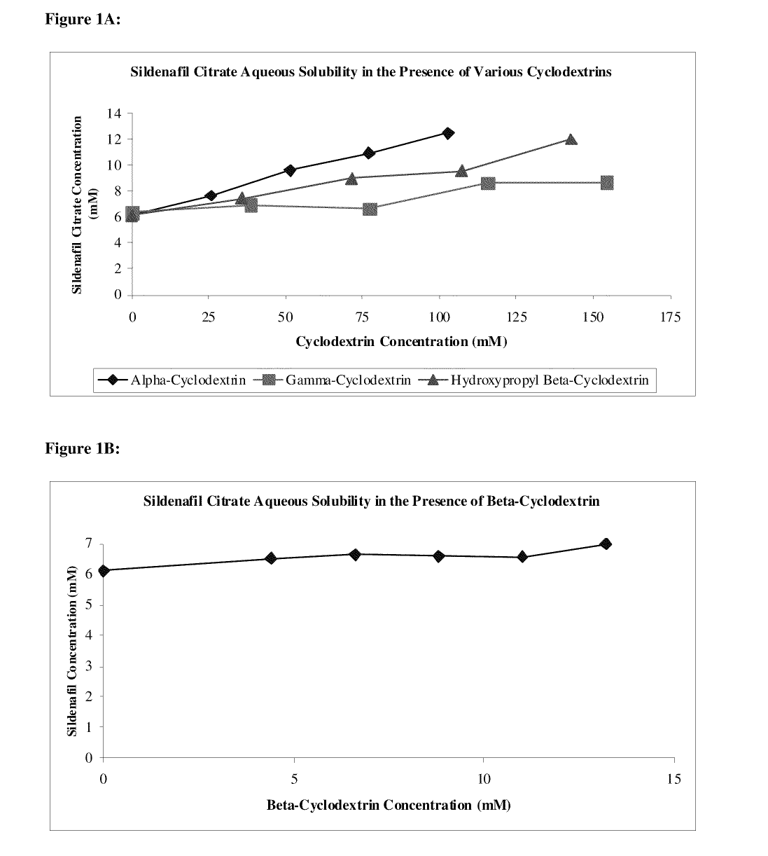

FIG. 1A depicts solubility data of sildenafil citrate complexed with alpha-cyclodextrin (ACD) at 0-100 mg/ml, gamma-cyclodextrin (GCD) at 0-200 mg/ml and hydroxy propyl-beta-cyclodextrin (HPBCD) at 0-200 mg/ml, and with no CD complexation in ddH₂O. Solutions were prepared by adding 50 mg of sildenafil citrate to ddH₂O samples with varying quantities of the different cyclodextrins. FIG. 1B depicts solubility data of sildenafil citrate complexed with beta-cyclodextrin (BCD) at 0-15 mg/ml These samples were shaken for 24 hours at 200 rpm. Excess sildenafil citrate was present in each of the vials for the duration of the solubility study. Following the shaking, the vial contents were filtered through 0.45 μm filters to remove any excess solute. The remaining solution was assayed by HPLC for sildenafil citrate content. The data of FIGS. 1A and 1B shows improved solubility of sildenafil citrate with all of the cyclodextrins evaluated and demonstrates that the aqueous solubility of sildenafil citrate is enhanced in the presence of cyclodextrins. The sildenafil citrate solubility enhancement in the presence of the various cyclodextrins was found to be ACD>HPBCD>GCD>>BCD.

### Example 2

### Solubility of Sildenafil Base Complexed With Cyclodextrin

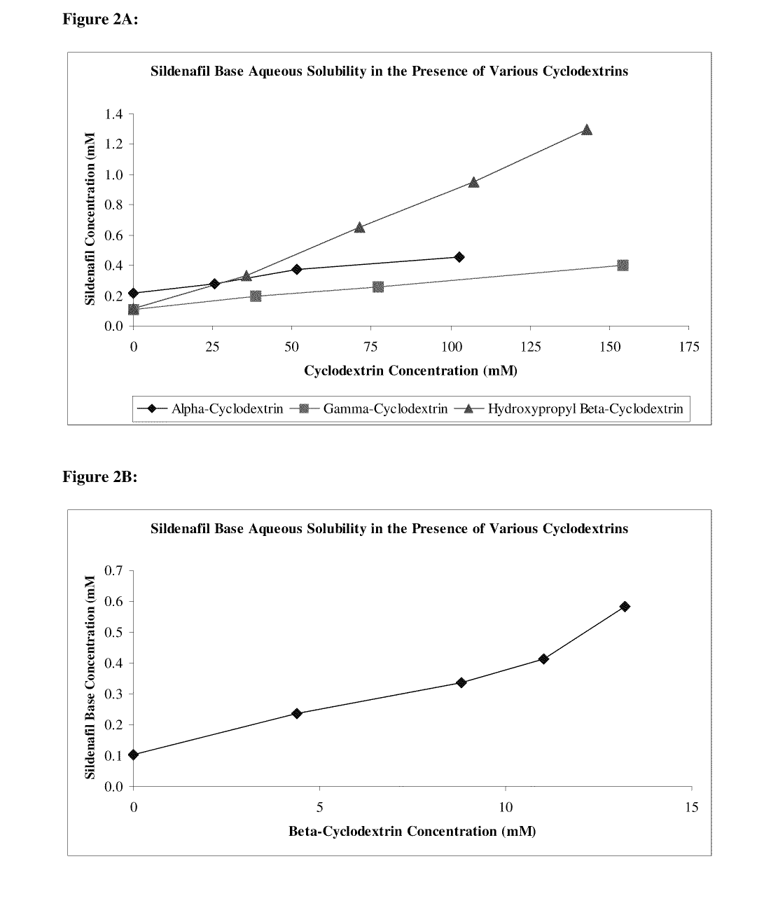

FIG. 2A depicts solubility data of sildenafil base complexed with alpha-cyclodextrin (ACD) at 0-100 mg/ml, gamma-cyclodextrin (GCD) at 0-200 mg/ml and hydroxy propyl-beta-cyclodextrin (HPBCD) at 0-200 mg/ml, and with no CD complexation in ddH₂O. Solutions were prepared by adding 25 mg sildenafil base to ddH₂O samples with varying quantities of the different cyclodextrins. FIG. 2B depicts solubility data of sildenafil base complexed with beta-cyclodextrin (BCD) at 0-15 mg/ml. These samples were shaken for 24 hours at 200 rpm. Excess sildenafil citrate was present in each of the vials for the duration of the solubility study. Following the shaking, the vial contents were filtered through 0.45 μm filters to remove any excess solute. The remaining solution was assayed by HPLC for sildenafil base content. The data of FIGS. 2A and 2B shows improved solubility of sildenafil base with all of the cyclodextrins evaluated and demonstrates that the aqueous solubility of sildenafil base is enhanced in the presence of cyclodextrins. The sildenafil base solubility enhancement in the presence of the various cyclodextrins was found to be HPBCD>BCD>ACD>GCD. For cyclodextrin concentrations below 50 mM, BCD presented with the greatest sildenafil base solubility enhancement. This is in agreement with the findings of Omani, et al. (2006).

### Example 3

### pH Dependence on the Solubility of Sildenafil Citrate Complexed with Cyclodextrin

Phosphate-citrate buffer (0.1M) solutions are prepared with pH's of 3.0; 4.0, 5.0; 6.0; 7.0 and 8.0 in ddH₂O (de-oxygenated water by purging with nitrogen gas). 4 ml of buffer with (alpha-cyclodextrin (100 mg/ml) and hydroxypropyl beta-cyclodextrin (200 mg/ml) or without cyclodextrin at the various pH values are placed in 4 ml vials and 50 mg sildenafil citrate is added to each vial. The vials are sealed and are shaken on an orbital shaker for 24 hours at 200 rpm. Excess sildenafil citrate was present in each of the vials for the duration of the solubility study. Following the shaking, the vial contents are filtered through 0.45 μm filters to remove any excess solute. The remaining solution is assayed by HPLC for sildenafil content. These results are presented in FIG. 3. The maximal aqueous solubility for sildenafil citrate appears to occur at pH 5. Alpha-cyclodextrin (ACD) presents with the greatest solubility enhancement.

### Example 4

### Preparation of Sildenafil Citrate Complexed with Alpha-Cyclodextrin

Sildenafil citrate (21.1 g) and alpha-cyclodextrin (60 g) are screened (50 mesh) and tumble mixed. The mixture is transferred to a mechanical kneader and dry mixed for 15 minutes. pH 5 phosphate-citrate buffer (0.1 M) (40 ml) is gradually added whilst the machine is kneading to produce a uniform paste. Kneading is continued for 2 hours ensuring a uniform paste-like consistency throughout the operation. Additional phosphate-citrate buffer is added, to maintain the paste-like consistency. The sildenafil-cyclodextrin paste is decanted from the kneader and oven dried at 40 DEG C. The dried mass is crushed with a mortar and pestle, after which it is passed through a 50 mesh screen. The product contains 27.1% m/m sildenafil citrate as determined by HPLC, resulting in a 104% sildenafil citrate recovery. The molecular composition of the product thus corresponds to 1 molecule sildenafil citrate and 1.85 molecules alpha-cyclodextrin.

### Example 5

### Preparation of Sildenafil Citrate Complexed with Alpha-Cyclodextrin

Sildenafil citrate (7.1 g); alpha-cyclodextrin (20 g) and 2.5 g N-vinyl-2-pyrrolidone are screened (50 mesh) and tumble mixed. The mixture is transferred to a mechanical kneader and dry mixed for 15 minutes. pH 5 phosphate-citrate buffer (0.1 M) (15 ml) is gradually added whilst the machine is kneading to produce a uniform paste. Kneading is continued for 2 hours ensuring a uniform paste-like consistency throughout the operation. Additional phosphate-citrate buffer is added, to maintain the paste-like consistency. The sildenafil-cyclodextrin paste is dried under vacuum at 40 DEG C. The dried mass is crushed to a fine powder in a ball mill, after which it is passed through a 50 mesh screen.

### Example 6

### Preparation of a Liquid Formulation Comprising Sildenafil Citrate Complexed with alpha-cyclodextrin

Sildenafil citrate solutions with concentrations of 3.5 mg/ml; 7 mg/ml and 14 mg/ml (equivalent sildenafil base concentrations of 2.5 mg/ml; 5 mg/ml and 10 mg/ml respectively) were produced in pH 5 phosphate-citrate buffer, whereby the sildenafil citrate formed an inclusion complex with alpha-cyclodextrin, were produced. Sildenafil citrate was complexed with 50 mg/ml; 80 mg/ml and 120 mg/ml of alpha-cyclodextrin dissolved in pH 5 phosphate-citrate buffer for 1.5 hours at 60 DEG C. Following complexation, samples were filtered through a 0.44 μm syringe filter and analyzed for sildenafil citrate concentration. Average sildenafil citrate recoveries from these samples of 101%; 102% and 99% respectively were obtained.

### Example 7

### Dissolution of an Inclusion Complex of Sildenafil Citrate Complexed With Alpha-Cyclodextrin Compared to a Physical Mixture of Sildenafil Citrate and Alpha-Cyclodextrin

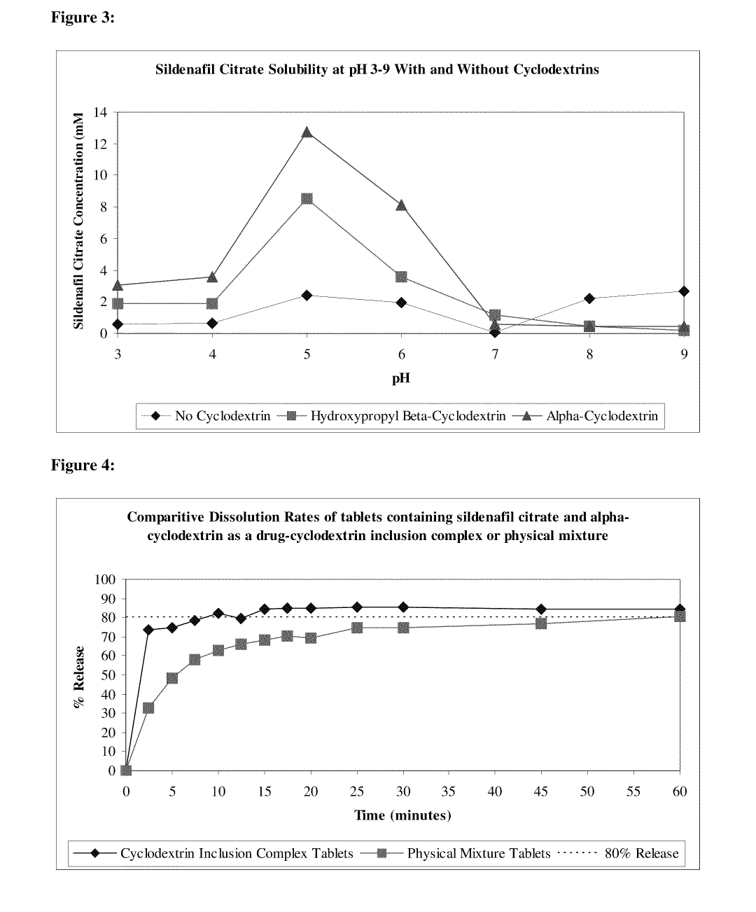

Dissolution behavior of the sildenafil citrate-alpha-cyclodextrin inclusion complex prepared in Example 4 and its corresponding physical mixture was evaluated in 0.01M HCl using USP Apparatus 2 (paddle, 50 rpm, 900 ml, 37 DEG C). Identical tablet formulations were used, employing the same pharmaceutical excipients for each, thus producing tablets containing an inclusion complex of sildenafil citrate-alpha-cyclodextrin obtained according to Example 4, as well as tablets containing a physical mixture of sildenafil citrate and alpha-cyclodextrin (non-complexed). The dissolution rates of the cyclodextrin inclusion complex and physical mixture with sildenafil citrate are shown in FIG. 4. The tablets produced with the sildenafil citrate-alpha-cyclodextrin inclusion complex prepared in Example 4 presented with a significantly faster dissolution rate compared to the corresponding physical mixture.

### Example 8

### Stability of an Inclusion Complex of Sildenafil Citrate Complexed with Alpha-Cyclodextrin

The powdered inclusion complex of sildenafil citrate and alpha-cyclodextrin prepared according to Example 4 were stored in an oven at 40 DEG C. Chromatographic analysis showed the complex to be chemically stable after one and three months storage at 40 DEG C.

### Example 9

### Preparation of a Solid Formulation Comprising an Inclusion Complex of Sildenafil Citrate Complexed with Alpha-Cyclodextrin

The inclusion complex of sildenafil citrate and alpha-cyclodextrin obtained according to the Example 5 was formulated into tablets with the following unit composition:

Sildenafil citrate-alpha-cyclodextrin complex (50 mg sildenafil base eq.): 270 mg;

Sodium hydrogen carbonate: 200 mg;

Microcrystalline cellulose: 94 mg;

Sodium hydroxymethylcellulose: 30 mg;

Magnesium stearate: 6 mg;

Total: 600 mg.

### Example 10

### Preparation of a Solid Formulation Comprising an Inclusion Complex of Sildenafil Citrate Complexed with Alpha-Cyclodextrin

The inclusion complex of sildenafil citrate and alpha-cyclodextrin obtained according to the Example 5 may be formulated into tablets with the following unit composition:

Sildenafil citrate-alpha-cyclodextrin complex (50 mg sildenafil base eq.):270 mg;

Microcrystalline cellulose: 106 mg;

Ac-di-sol: 20 mg;

Magnesium stearate: 4 mg;

Total: 400 mg.

The Ac-di-sol and microcrystalline cellulose are premixed in a blender. The sildenafil citrate-alpha-cyclodextrin complex is added to the mixture and blended. The magnesium stearate is screened in and blended. The mixture is compressed into tablets.

### Example 11

### Bioavailability of an Inclusion Complex of Sildenafil Citrate Complexed with Alpha-Cyclodextrin Compared to a Sildenafil Citrate Suspension

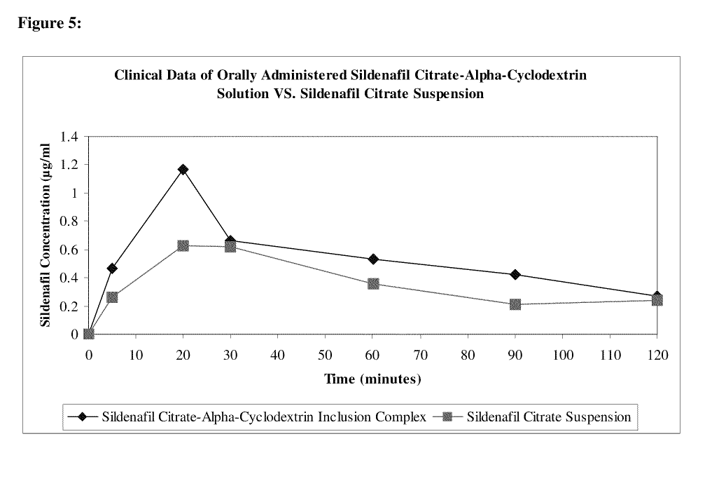

The inclusion complex of sildenafil citrate and alpha-cyclodextrin prepared according to Example 6 to yield a sildenafil citrate concentration of 14 mg/ml (equivalent of 10 mg/ml sildenafil base) was evaluated in a bioavailability study in a rat model vs. a 14 mg/ml sildenafil citrate suspension in pH 5 phosphate-citrate buffer. 24 Rats, 12 per formulation, were fasted over night and dosed (30 mg/kg) by oral gavage with the formulations. At predetermined intervals, blood samples were taken, which were assayed for sildenafil content. The results from this clinical study are presented in FIG. 5. These results clearly show that the sildenafil citrate administered as an inclusion complex, presents with greater bioavailability, as well as greater Cₘₐₓ, which is double that of the sildenafil citrate suspension formulation.

What is claimed is:

1. An inclusion complex comprising a sildenafil salt and alpha-cyclodextrin.

2. The inclusion complex of claim 1, wherein the sildenafil salt is sildenafil-citrate.

3. The inclusion complex of claim 1, wherein the molar ratio of the sildenafil salt to alpha-cyclodextrin is from 1:1 to 1:20, inclusive.

4. The inclusion complex of claim 3, wherein the molar ratio of the sildenafil salt to alpha-cyclodextrin is from 1:1 to 1:5, inclusive.

5. The inclusion complex of claim 4, wherein the molar ratio of the sildenafil salt to alpha-cyclodextrin is from 1:1 to 1:2.5, inclusive.

6. The inclusion complex of claim 1, wherein the solubility of the sildenafil salt upon dissolution of the complex in deionized water at 20° C. is increased by at least 1.5-fold compared to the solubility of sildenafil salt in uncomplexed form.

7. The inclusion complex of claim 6, wherein the solubility of the sildenafil salt is increased by at least 2-fold.

8. The inclusion complex of claim 1, wherein the solubility of the sildenafil salt upon dissolution of the complex in deionized water at 20° C. is at least 8 mM.

9. The inclusion complex of claim 8, wherein the solubility of the sildenafil salt is at least 10 mM.

10. The inclusion complex of claim 9, wherein the solubility of the sildenafil salt is at least 12 mM.

11. A formulation comprising a sildenafil salt, alpha-cyclodextrin, and a carrier optionally selected from the group consisting of a complexing agent, a filler, a diluent, a granulating agent, a disintegrant, a lubricant, and a glidant.

12. The formulation of claim 11, wherein the carrier is one or more of microcrystalline cellulose, glucose, sodium lauryl sulphate, crosscarmellose sodium, colloidal silica, talc, magnesium stearate, sodium benzoate, aluminum magnesium silicate, lactose, a dye, or a polymer-based film coating.

13. The formulation of claim 11, wherein the molar ratio of alpha-cyclodextrin to sildenafil salt is greater than 1:1, inclusive.

14. The formulation of claim 13, wherein the molar ratio of alpha-cyclodextrin to sildenafil salt is greater than 5:1, inclusive.

15. The formulation of claim 14, wherein the molar ratio of alpha-cyclodextrin to sildenafil salt is greater than 10:1, inclusive.

16. A formulation comprising the inclusion complex of claim 1 and a carrier.

17. A formulation comprising an effective amount of the inclusion complex of claim 1 and a carrier.

18. The formulation of claim 11, wherein the carrier is a pharmaceutically acceptable carrier.

19. The formulation of claim 11, wherein the formulation is a solid.

20. The formulation of claim 11, wherein the formulation is a liquid.

21. The formulation of claim 20, wherein the pH is less than about 8.0 at 20° C.

22. The formulation of claim 20, wherein the pH is between about 4.0 and 6.0 at 20° C.

23. The formulation of claim 11, wherein the sildenafil salt is present in an amount between about 0.1 mg and about 200 mg sildenafil, inclusive.

24. The formulation of claim 23, wherein the sildenafil salt is present in an amount between about 20 mg and about 100 mg sildenafil, inclusive.

25. The formulation of claim 24 wherein the sildenafil salt is present in an amount of about 25 mg sildenafil.

26. The formulation of claim 24 wherein the sildenafil salt is present in an amount of about 50 mg sildenafil.

27. The formulation of claim 24 wherein the sildenafil salt is present in an amount of about 100 mg sildenafil.

28. A substantially pure form of the inclusion complex of claim 1.

29. A method of treating erectile dysfunction in an individual, comprising administering to the individual an effective amount of the complex of claim 1.

30. A method of treating erectile dysfunction in an individual, comprising administering to the individual an effective amount of the formulation of claim 11.

31. A method of treating pulmonary hypertension in an individual, comprising administering to the individual an effective amount of the complex of claim 1.

32. A method of treating pulmonary hypertension in an individual, comprising administering to the individual an effective amount of the formulation of claim 11.

33. The method of claim 29, wherein the complex is administered parenterally.

34. The method of claim 31, wherein the complex is administered parenterally.

35. The method of claim 29, wherein the complex is administered orally.

36. The method of claim 31, wherein the complex is administered orally.

37. The method of claim 29, wherein the dosage of sildenafil salt administered is between about 0.1 mg and about 200 mg sildenfil, inclusive.

38. The method of claim 31, wherein the dosage of sildenafil salt is between about 0.1 mg and about 200 mg sildenafil, inclusive.

39. The method of claim 29, wherein the dosage of sildenafil salt is about 25, 50, or 100 mg sildenafil.

40. The method of claim 31, wherein the dosage of sildenafil salt is about 25, 50, or 100 mg sildenafil.

41. A method of inhibiting cyclic guanosine monophosphate (cGMP)-specific phosphodiesterase 12 type-5 (PDE5) enzyme, comprising contacting the PDE5 enzyme with an effective amount of the sildenafil salt of the inclusion complex of claim 1.

42. A kit for the treatment of erectile dysfunction, comprising an inclusion complex of claim 1; and instructions for use.

43. A kit for the treatment of pulmonary hypertension, comprising an inclusion complex of claim 1; and instructions for use.

44. A method of producing an inclusion complex of claim 1, comprising admixing the sildenafil salt with alpha-cyclodextrin.

45. The method of claim 44, further comprising adding a solvent, mixed solvent, or buffer to the sildenafil salt, alpha-cyclodextrin, and/or mixture thereof.

46. A method of producing an inclusion complex of claim 1, comprising the steps of:
a. admixing the sildenafil salt and alpha-cyclodextrin; and
b. adding a suitable amount of solvent, mixed solvent, or buffer to the mixture of step (a) and mixing until a suspension or solution is formed.

47. The method of claim 45, wherein the solvent, mixed solvent, or buffer is a buffer.

48. The method of claim 47, wherein the buffer is a phosphate-citrate buffer.

49. The method of claim 45, wherein the solvent, mixed solvent, or buffer has a pH between about 1.0 and about 6.0.

50. The method of claim 49, wherein the solvent, mixed solvent, or buffer has a pH of about 5.0.

51. The method of claim 45, wherein the solvent, mixed solvent, or buffer is heated to greater than 40° C.

52. The method of claim 51, wherein the solvent, mixed solvent, or buffer is heated to greater than 50° C.

53. The method of claim 52, wherein the solvent, mixed solvent, or buffer is heated to greater than 60° C.

54. The method of claim 46, wherein step (a) further comprises admixing a suitable polymer.

55. The method of claim 54, wherein the suitable polymer is selected from polyvinylpyrrolidone, hydroxypropyl methylcellulose, carboxymethylcellulose, and Plasdone® Povidone.

56. The method of claim 46, wherein mixing is continued for at least 0.2 hr following formation of the suspension or solution.

57. The method of claim 56, wherein mixing is continued for at least 0.5 hr following formation of the suspension or solution.

58. The method of claim 46, further comprising a step for drying the product of step (b).

59. The method of claim 58, wherein drying the product of step (b) comprises evaporation.

60. The method of claim 59, wherein the evaporation occurs for greater than about 0.1 hour.

61. The method of claim 59, wherein the evaporation is conducted under vacuum.

62. The method of claim 59, wherein the evaporation is conducted under atmospheric pressure.

63. The method of claim 58, wherein drying the product of step (b) comprises spray-drying.

64. The method of claim 58, wherein drying the product of step (b) comprises freeze-drying.

65. The method of claim 58, wherein drying the product of step (b) comprises spray-granulation.

66. A method for improving the solubility of a sildenafil salt in water comprising complexing the sildenafil salt with alpha-cyclodextrin.

67. The method of claim 66, wherein the sildenafil salt is sildenafil citrate.

68. The method of claim 66 wherein the solubility of the sildenafil salt in deionized water at 20° C. is increased by at least 1.5-fold compared to the solubility of sildenafil salt in uncomplexed form.

69. The method of claim 68, wherein the solubility of the sildenafil salt is increased by at least 2-fold.
